(19) United States
(12) Patent Application Publication
(10) Pub. No.: US 2010/0174135 A1
(43) Pub. Date: Jul. 8, 2010

(54) Synchronized relaxing systems and methods

(76) Inventor: Youngtack Shim, Port Moody (CA)

Correspondence Address: Youngtack Shim, 155 Aspenwood Drive, Port Moody, BC, V3H 5A5, CA

(21) Appl. No.: 12/318,662

(22) Filed: Jan. 6, 2009

(51) Int. Cl. A61F 5/00; A61B 5/103; A61B 19/00

(52) U.S. Cl. 600/38; 600/587; 128/898

(57) ABSTRACT

The present invention generally relates to pelvic relaxing systems capable of obtaining various audiovisual and/or control signals and synchronizing various relaxing operations of the systems with such signals. More particularly, the present invention relates to various pelvic relaxing systems which obtain pre-recorded and/or real-time audiovisual (or control) signals from an user and/or a third party, analyze various bases carried by such signals, and synchronize various operations of such systems with one or more of such signals. Therefore, the pelvic relaxing systems may provide various stimuli to one or multiple portions of a pelvic structure based on temporal and/or spatial synchronization with such signals. Conversely, the present invention relates to various pelvic relaxing systems which may generate audiovisual signals synchronized with various operations performed by such systems. The present invention relates to various methods of obtaining such signals from various sources, obtaining such signals through combining and/or synchronizing multiple raw signals, and synchronizing various operations thereof with such signals. The present invention further relates to various processes for making such members, units, and parts of the pelvic relaxing systems capable of obtaining the signals and synchronizing various operations thereof with such signals.

### CROSS-REFERENCES TO RELATED APPLICATIONS

The present application claims an earlier invention date of the Disclosure Document entitled the same, deposited in the U.S. Patent and Trademark Office (the “Office”) on Jan. 12, 2007 under the Disclosure Document Deposit Program (“DDDP”) of the Office, and bearing the Serial Number 611,027. The present application also claims earlier invention dates of other Disclosure Documents, the first of which is entitled “Dynamic control relaxing systems and methods,” deposited in the Office on Jan. 12, 2007 under the DDDP, and bearing the Serial Number 611,023, and the second of which is entitled “Audio relaxing systems and methods, deposited in the Office on Jan. 23, 2007 under the DDDP, and bearing the Serial Number 611,331. It is to be appreciated that an entire portion of each of the above Disclosure Documents is incorporated herein by reference.

### CROSS-REFERENCES TO RELATED APPLICATIONS

The present application claims an earlier invention date of the Disclosure Document entitled the same, deposited in the U.S. Patent and Trademark Office (the “Office”) on Jan. 12, 2007 under the Disclosure Document Deposit Program (the “DDDP”) of the Office, and which bears the Serial Number 611,027 an entire portion of which is incorporated herein by reference.

### FIELD OF THE INVENTION

The present invention generally relates to pelvic relaxing systems capable of obtaining various audiovisual and/or control signals and synchronizing various relaxing operations of the systems with such signals. More particularly, the present invention relates to various pelvic relaxing systems which obtain pre-recorded and/or real-time audiovisual (or control) signals from an user and/or a third party, analyze various bases carried by such signals, and synchronize various operations of such systems with one or more of such signals. Therefore, the pelvic relaxing systems may provide various stimuli to one or multiple portions of a pelvic structure based on temporal and/or spatial synchronization with such signals. Conversely, the present invention relates to various pelvic relaxing systems which may generate audiovisual signals synchronized with various operations performed by such systems. The present invention relates to various methods of obtaining such signals from various sources, obtaining such signals through combining and/or synchronizing multiple raw signals, and synchronizing various operations thereof with such signals. The present invention further relates to various processes for making such members, units, and parts of the pelvic relaxing systems capable of obtaining the signals and synchronizing various operations thereof with such signals.

### BACKGROUND OF THE INVENTION

A vaginal anatomy typically includes a vaginal entry and a vaginal wall, where such an entry defines an orifice therethrough, while the wall includes muscles and defines a vaginal cavity which extends inwardly from the entry and which is also bound by such muscles. The entry forms a clitoris thereon, and a paraurethral gland of an urethral sponge of a clitoris (also called the Grafenberg spot or G spot) is believed to be defined on the wall. The vaginal wall is formed essentially of two sets of muscles, the former extending longitudinally while the latter encircling the vagina. These muscles are specifically termed as “pubococygenus” and “levator ani” and are located immediately adjacent to the vagina. These muscles have general appearance of a hammock with its two ends connected to the sides of a pelvis. In particular, the pubococygenus is basically a sphincter muscle which passes through a middle third of the vagina and runs in a circular band, with a ring-like ridges forming a part of a urethra and anus. For simplicity of illustration, this vaginal anatomy is to be referred to as “a pelvic structure,” the vaginal entry as an “entry,” and the vaginal wall as a “wall.” In addition, such a pelvic structure is referred to as the “standard pelvic structure” or simply the “pelvic structure” hereinafter for simplicity of illustration, unless otherwise specified.

Among devices currently available in the market for enhancing sexual functioning are dildos, vaginal exercise bars, and prostate stimulators. These devices generally provide stimuli from friction upon manipulation of their stimulators or by pressure due to distension of the pelvic cavity effected by a volume of such stimulators.

For example, manual pelvic relaxing devices have been proposed in various configurations as disclosed in various prior art such as, e.g., U.S. Pat. No. 3,996,930 to Sekulich, U.S. Pat. No. 5,690,603 to Kain, U.S. Pat. No. 5,690,604 to Barnett, U.S. Pat. No. 5,853,362 to Jacobs, U.S. Pat. No. 6,203,491 to Uribe, U.S. Pat. App. Pub. No. 2005/0187431 by Hudson, U.S. Pat. App. Pub. No. 2005/0228218 by Skidmore et al., U.S. Pat. No. 6,540,667 to Hickman, and the like. Being manual, users have to manually move such devices in and out of the pelvic cavity and/or around the pelvic opening.

In order to overcome inconvenience thereof, various automatic mechanisms have been added to the pelvic relaxation devices. In one class of examples, electric motors or electromagnetic vibration mechanisms have been incorporated to various automatic devices for effecting vibration as disclosed in various prior art such as, e.g., U.S. Pat. No. 3,451,391 to Tavel, U.S. Pat. No. 3,504,665 to Bakunin et al., U.S. Pat. No. 3,626,931 to Bysakh, U.S. Pat. No. 3,669,100 to Csanad, U.S. Pat. No. 3,991,751 to O'Rourke, U.S. Pat. No. 4,788,968 to Rudashevsky et al., U.S. Pat. No. 5,067,480 to Woog et al, U.S. Pat. No. 6,056,705 to Stigar-Brown, U.S. Pat. Appl. Pub. 2004/0034315 to Chen, U.S. Pat. Appl. Pub. 2004/0127766 to Chen, and the like.

In another class of example, automatic pelvic relaxing devices have used various mechanisms of converting rotational movements generated by such electric motors into translational movements for effecting horizontal and/or vertical translation of their stimulators. Several examples of such prior art include U.S. Pat. No. 4,722,327 to Harvey, U.S. Pat. No. 4,790,296 to Segal, U.S. Pat. No. 5,076,261 to Black, U.S. Pat. No. 5,725,473 to Taylor, U.S. Pat. No. 6,142,929 to Padgett, U.S. Pat. No. 6,422,993 to Hudson, U.S. Pat. No. 6,866,645 to Lee, and U.S. Pat. Appl. Pub. 2004/0147858

Various automatic pelvic relaxing devices have also used various mechanisms for converting rotational movements generated by such electric motors into lateral movements for effecting horizontal translation of their stimulators along a direction generally normal to axes of such stimulators. Several examples of such prior art are U.S. Pat. No. 5,460,597 to Hopper, U.S. Pat. No. 5,470,303 to Leonard et al., and U.S. Pat. No. 5,851,175 to Nickell.

Other therapeutic devices, although developed for various purposes other than pelvic relaxing, seem to have been used as alternatives as such conventional pelvic relaxing devices. In one class of examples, various manual or vibration devices have been disclosed to train or heal pelvic muscles as exemplified in U.S. Pat. No. 3,598,106 to Buning, U.S. Pat. No. 4,241,912 to Mercer et al., and U.S. Pat. No. 4,574,791 to Mitchener. In another class of examples, various devices have been developed for massaging various portions of a human body as disclosed in U.S. Pat. No. 4,055,170 to Nohmura, U.S. Pat. No. 4,825,853 to Iwamoto et al., U.S. Pat. No. 4,846,158 to Teranishi, U.S. Pat. No. 4,911,149 to Borodulin et al., U.S. Pat. No. 5,063,911 to Teranishi, and the like. In another class, massage devices have also been devised to provide translational movements as disclosed in U.S. Pat. No. 4,002,164 to Bradley, U.S. Pat. No. 5,085,207 to Fiore, U.S. Pat. No. 5,676,637 to Lee, and the like. In another class of examples, various devices have also been arranged to provide rotating, tapping, swinging and/or swivelling movements as described in U.S. Pat. No. 4,162,675 to Kawada, U.S. Pat. No. 6,632,185 to Chen, U.S. Pat. No. 4,088,128 to Mabuchi, U.S. Pat. No. 4,513,737 to Mabuchi, U.S. Pat. No. 4,827,914 to Kamazawa, U.S. Pat. No. 4,834,075 to Guo et al., U.S. Pat. No. 5,183,034 to Yamasaki et al., and U.S. Pat. No. 6,402,710 to Hsu. A vacuum device of U.S. Pat. No. 4,033,338 to Igwebike as well as a balloon device of U.S. Pat. No. 4,050,449 to Castellana et al. have also been proposed.

Regardless of their detailed mechanisms and/or movements effected thereby, all of these prior art devices suffer from common drawbacks. Excluding those manual ones, typical automatic devices consist of main modules and control modules which operatively couple with the main modules by wire for delivering electric power and control signals. Such wire, however, tends to be easily tangled and damaged. To overcome this defect, modern automatic pelvic relaxing device are fabricated as single unitary articles each with a main body and a handle which fixedly couples with a top part of the main body. The main body is generally designed to be inserted into the pelvic cavity, whereas the handle is shaped and sized to provide a grip for the user and also incorporates therein various control buttons. Accordingly, the handle consists of a space to form the grip and another space for such buttons. In order to avoid providing an inadvertently long device, however, a part of the handle closer to the main body is recruited to define the grip, whereas the rest of the handle houses the control buttons. It is to be appreciated, however, that all control buttons of conventional automatic devices are either on/off switches or speed control switches, where the on/off switches turn on and off the entire device or a specific movement thereof, and the speed control switches control a speed of the specific movement. In addition, such switches are typically designed to be activated and deactivated each time the user presses or touches them. Accordingly, when the user inadvertently touches any of such on/off and control switches during use, the device may be accidentally turned off, change speeds, and the like. In order to avoid such inadvertent operation, the control buttons have been incorporated as far away from the grip space of the handle, which in turn causes the very inconvenience of requiring the user to change the grip or to move his or her hand to manipulate the control buttons during operation when the user wants to change the speed of movement.

In contrary to these devices, novel pelvic relaxing systems, methods, and/or processes have already been conceived of and disclosed in numerous co-pending Applications of the same Applicant. For example, various pelvic relaxing systems have been proposed for manipulating their input and/or sensor units without requiring the user to change the grip, for playing sounds while effecting various movements of such parts onto such a portion of the pelvic structure, for providing various stimuli to the clitoris and/or G-spot of the user, for providing interactive capabilities thereto, for synchronizing movements of their various parts and/or operations thereof with internal and/or external signals, for incorporating electric stimulators thereinto, for installing the body members capable of adjusting their configurations, for incorporating retention mechanisms thereinto, for providing feedback mechanisms thereto, for generating reciprocating movements of only portions of their body members, and the like. Although these novel systems solve most deficiencies of the conventional devices, none of them are capable of synchronizing their operations with various audiovisual and/or control signals.

Therefore, there is a need for a pelvic relaxing system capable of synchronizing its operations with various audiovisual and/or control signals supplied thereto internally and/or externally so that the system may effect various movements of its parts and provide various stimuli to such portions based upon the signals. There also is a need for a pelvic relaxing system capable of generating audiovisual signals based upon operations of various members of the system so that the system may generate a sound and/or an image based upon various movements of such parts and/or various stimuli provided to such portions. In addition, there is a need for a pelvic relaxing system capable of synchronizing its operations with such signals without requiring the user to move the entire system.

### SUMMARY OF THE INVENTION

The present invention generally relates to pelvic relaxing systems capable of obtaining various audiovisual (or control) signals and synchronizing various relaxing operations of such systems with such signals. More particularly, the present invention relates to various pelvic relaxing systems which obtain pre-recorded and/or real-time audiovisual (or control) signals from an user and/or a third party, analyze various bases carried by such signals, and synchronize various operations of such systems with one or more of such bases. Therefore, such pelvic relaxing systems may provide various stimuli onto one or multiple portions of the pelvic structure in temporal and/or spatial synchronization with the signals. The systems may also preferably effect movements of various parts thereof, effect various movements of a single part or multiple parts thereof, and/or provide such stimuli to various portions of the pelvic structure without requiring an user to reciprocate, rotate, and/or otherwise move the entire system. Accordingly, the user may receive such stimuli which are synchronized with various bases carried by such signals without having to manually imitate the sounds and/or images of such signals. Conversely, the present invention also relates to various pelvic relaxing systems which may generate audiovisual signals which may be synchronized with various operations of the systems. Accordingly, the user may listen to the sounds and/or watch the images by audiovisual output units during manual operation of the system such as, e.g., movements of such parts of the system and/or entire system, various features of such movements, and the like.

The present invention also relates to various methods of obtaining the audiovisual (or control) signals from various sources, obtaining such compound and/or synchronized signals by manipulating such audiovisual (or control) signals, and synchronizing various operations of the system with such signals. Conversely, the present invention relates to various methods of effecting a single movement or multiple movements of one or multiple parts the system by one or multiple actuator members thereof, alternatively generating manual movements of an entire system, and generating sounds and/or images synchronized with one or more of such movements. The present invention further relates to various processes for providing the above system and various members, units, and/or parts thereof capable of obtaining the signals and synchronizing various operations of such a system with the signals. The present invention also relates various processes for providing such a system and various members, units, and/or parts thereof capable of generating the sounds and/or images synchronized with one or more of such movements of the parts of the system and/or those of the entire system.

Therefore, one objective of the present invention is to provide a pelvic relaxing system which obtains various audiovisual (or control) signals and performs its operations based upon such signals. Another objective of this invention is to provide a pelvic relaxing system which obtains the signals and temporally and spatially synchronizes its operations with the signals. Conversely, a related objective of the present invention is to provide a system which performs its operations while generating various sounds and/or images which are based upon or synchronized with such operations. In both of these objectives, such signals may be pre-recorded signals or may instead be provided in real time. In both of these objectives, such signals may be generated by, may be provided from, may contain therein or may be related to an user of the system and/or a third party.

Another objective of the present invention is to provide another pelvic relaxing system which obtains various audiovisual (or control) signals and effects multiple movements of a single part of its body member one at a time (or simultaneously) while synchronizing each of the movements with such signals. Conversely, a related objective of the present invention is to provide a system which effects multiple movements of its single part one at a time (or simultaneously) while generating various sounds and/or images based thereupon or in synchronization therewith.

Another objective of the present invention is to provide another pelvic relaxing system which obtains various audiovisual (or control) signals and effects identical, similar or different movements of multiple parts of its body member one at a time (or simultaneously) based upon such signals. Another objective of this invention is to form a system which obtains such signals and effects identical, similar or different movements of its multiple parts one at a time (or simultaneously) while synchronizing such movements with such signals. Conversely, a related objective of this invention is to provide a system effecting identical, similar or different movements of its multiple parts simultaneously (or one at a time) while generating various sounds and/or images based thereupon or in synchronization therewith.

Another objective of the present invention is to provide another pelvic relaxing system which obtains various audiovisual (or control) signals and provides identical, similar or different stimuli onto a single or multiple portions of such a pelvic structure one at a time (or simultaneously) based upon the signals. Another objective of the present invention is to form a system which obtains the signals and provides identical, similar or different movements of such multiple portions of the pelvic structure one at a time (or simultaneously) while synchronizing the stimuli with such signals. Conversely, a related objective of this invention is to provide a system which provides identical, similar or different stimuli to multiple portions of such a pelvic structure one at a time (or simultaneously) while generating various sounds and/or images based thereupon or in synchronization therewith.

Another objective of the present invention is to provide another pelvic relaxing system which obtains various audiovisual (or control) signals and effects horizontal or vertical translation of a single part or multiple parts of its body member, angled or transverse translation thereof, rotation or pivoting thereof, and/or configurational change thereof one at a time (or simultaneously) while synchronizing each of such movements and/or changes with such signals. Conversely, a related objective of the present invention is to provide a system which effects one or more of the above movements and/or changes one at a time (or simultaneously) while generating various sounds and/or images based on one or more of such movements or in synchronization therewith.

Another objective of the present invention is to provide another pelvic relaxing system which obtains internal audiovisual (or control) signals stored therein and perform its operation based upon or in synchronization with such signals. Conversely, a related objective of this invention is to provide a system which performs its operations while playing various sounds and/or displaying various images generated from such signals based upon such operations or in synchronization therewith.

Another objective of the present invention is to provide another pelvic relaxing system which obtains external audiovisual (or control) signals from an external audiovisual or communication device or through an internet and perform its operation based upon or in synchronization with such signals. Conversely, a related objective of this invention is to provide a system which performs its operations while playing various sounds and/or displaying various images generated from the signals based on such operations or in synchronization therewith.

Another objective of the present invention is to provide another pelvic relaxing system which obtains various audiovisual (or control) signals, generates a compound signal through superposing or combining two or more of such audiovisual (or control) signals, and perform its operation based upon or in synchronization with the compound signal. Conversely, a related objective of this invention is to provide another system which performs its operations while playing various sounds and/or displaying various images generated from the compound signal based upon the operations or in synchronization therewith.

Another objective of the present invention is to provide another pelvic relaxing system which obtains various audiovisual (or control) signals each defining a content basis, a voice basis, an action basis, and/or a background basis, and perform its operation based upon or in synchronization with at least one of such bases. Conversely, a related objective of the present invention is to provide such a system which performs its operations while playing various sounds and/or displaying various images which are generated from such signals based upon at least one of such bases or in synchronization therewith.

Another objective of the present invention is to provide another pelvic relaxing system which obtains various audiovisual (or control) signals, generates a synthesized signal by changing at least one of their content bases, voice bases, action bases, and/or background bases of such audiovisual (or control) signals, and perform its operation based upon or in synchronization with the synthesized signal. Conversely, a related objective of this invention is to provide another system which performs its operations while playing various sounds and/or displaying various images generated from such a synthesized signal based upon the operations or in synchronization therewith.

Another objective of the present invention is to provide another pelvic relaxing system which obtains various audiovisual (or control) signals and turns on (and off) itself, activates (and terminates) its actuator and/or control members, and/or initiates (and stops) its relaxing operations based upon or in synchronization with such signals. Conversely, a related objective of this invention is to provide a system which turns on (and off) itself, activates (and terminates) its actuator and/or control members, and/or initiates (and stops) its relaxing operations while playing such sounds and/or displaying such images based on such operations or in synchronization therewith.

Another objective of the present invention is to provide another pelvic relaxing system which obtains various audiovisual (or control) signals representing a disposition or insertion of an insertable part of the system with respect to the pelvic structure, and then performs its operation based upon or in synchronization with such signals. Conversely, a related objective of this invention is to provide a system which performs its operations while playing various sounds and/or displaying various images generated from such signals based on such disposition or insertion of the insertable part thereof or in synchronization therewith.

Another objective of the present invention is to provide another pelvic relaxing system which obtains various audiovisual (or control) signals which represent various dynamic features of overall movements of the system with respect to the pelvic structure, and performs its operation based upon or in synchronization with such signals. Conversely, a related objective of this invention is to provide another system which performs its operations while playing various sounds and/or displaying various images generated from the above signals based on such disposition or insertion of the insertable part thereof or in synchronization therewith.

Another objective of the present invention is to provide another pelvic relaxing system which obtains various audiovisual (or control) signals representing verbal and/or bodily commands from the user and/or third party, and performs its operation based upon or in synchronization with the signals. Conversely, a related objective of this invention is to provide a system which performs its operations while playing various sounds and/or displaying various images which are generated based on such verbal and/or bodily commands or in synchronization therewith.

Various aspects and/or embodiments of various systems, methods, and/or processes of this invention will now be described, where such aspects and/or embodiments only represent different forms. Such systems, methods, and/or processes of this invention, however, may also be embodied in many other different forms and, therefore, should not be limited to the aspects and/or embodiments which are set forth herein. Rather, various exemplary aspects and/or embodiments described herein are provided so that this disclosure will be thorough and complete, and fully convey the scope of the present invention to one of ordinary skill in the art. It is to be understood that various movements and mechanisms therefor as well as various control algorithms of the prior art devices as described in the above Background of the Invention are to be incorporated herein in their entireties by reference.

In one aspect of the present invention, a pelvic relaxing system may provide tactile stimuli onto at least a portion of a pelvic structure at least partially based upon at least one audiovisual (or control) signal, where the pelvic structure may include an entry and a wall, where the entry may be arranged to define an orifice therethrough and where the wall may be arranged to include muscles and to form an internal cavity extending inwardly from the entry and bound by the muscles. This pelvic structure is to be referred to as the “standard pelvic structure” or simply as the “pelvic structure” throughout the disclosure unless otherwise specified.

In one exemplary embodiment of this aspect of the invention, a system may include at least one body member, at least one actuator member, and at least one control member. The body member may have a first part for contacting the portion of the pelvic structure when engaged therewith as well as a second part for providing a grip to an user, where such a body member is to be referred to as “the body member of the first type” throughout this disclosure. The actuator member may be arranged to effect at least one movement of the first part for providing the stimuli onto the portion of the structure through the movement, where such an actuator member is to be referred to as the “actuator member of the first type” throughout this disclosure. The control member may be arranged to include at least one of at least one sensor unit and at least one input unit therein, to receive an user input defining at least one dynamic pattern, and to effect the movement of the first part at least partially based on the dynamic pattern of the user input. Such a control member is to be referred to as the “control member of the first type” throughout this disclosure.

In one example, such a control member may also obtain the signal from at least one source and manipulate the actuator and/or body members based upon the signal during use of such a system. In another example, the control member may obtain at least one signal from such multiple signals based upon the user input and manipulate the actuator and/or body members based upon the obtained signal.

In another example, the control member may have a main switch for operating between multiple states each defining a different operation and/or state of the actuator member, obtain at least one signal from such multiple signals based on the state of the main switch, and manipulate at least one of such body and actuator members based upon the obtained signal. In another example, the control member may obtain at least one signal from such multiple signals based upon insertion (or an extent of insertion) of the first unit into the internal cavity, and manipulate the body and/or actuator members based upon the obtained signal. In another example, the control member may also obtain at least one signal from such multiple signals based on another movement of the system as a whole and manipulate the body and/or actuator members based on the obtained signal.

In another exemplary embodiment of this aspect of the invention, a system may include at least one body member of the first type, at least one actuator member of the first type, at least one control member of the first type, and the like. In one example, such a control member may include at least one storage unit therein for storing multiple the signals which may be pre-recorded and related to the user and/or a third party, obtain at least one signal from the storage unit, and manipulate the actuator and/or body members based upon (or in at least partial synchronization with) the retrieved signal. In another example, the control member may instead obtain from an external audiovisual device at least one of such signals which may be pre-recorded and related to the user and/or a third party, and manipulate the body and/or actuator members based on (or in at least partial synchronization with) the obtained signal. In another example, the control member may obtain from an external communication device at least one of such signals which may be pre-recorded or real time and also related to a third party, and manipulate the body and/or actuator members based on (or in at least partial synchronization with) the obtained signal. In another example, the control member may obtain through an internet at least one of such signals which may be pre-recorded or real time and related to a third party, and then manipulate the body and/or actuator members based on (or in at least partial synchronization with) the obtained signal. In another example, the control member may obtain at least two of such signals which may be pre-recorded or real time and also related to the user and/or a third party, form a compound signal by combining such at least two signals, and manipulate the body and/or actuator members based on (or in at least partial synchronization with) the compound signal. In another example, the control member may obtain a first signal defining a first content basis and a first voice basis from such multiple signals which may be pre-recorded or real time and related to the user and/or a third party, obtain a second signal defining a second content basis and a second voice basis from such multiple signals, generate at least one synthesized signal by replacing one of such bases of one of the first and second signals by such one of the bases of another of such first and second signals, and manipulate the body and/or actuator members based upon (or in at least partial synchronization with) the synthesized signal. In another example, the control member may obtain a first signal defining a preset content basis as well as a preset voice basis from such multiple signals which may be pre-recorded or real time and related to the user and/or a third party, alter at least one of such bases of the first signal, and manipulate the body and/or actuator members based on (or in at least partial synchronization with) the altered signal.

In another aspect of the present invention, a pelvic relaxing system may provide tactile stimuli onto at least a portion of the standard pelvic structure through at least one movement of at least a part of the system based on (or in synchronization with) at least one audiovisual (or control) signal.

In one exemplary embodiment of this aspect of the invention, a system may include at least one body member of the first type, at least one actuator member, as well as at least one control member of the first type. The actuator member may then be arranged to effect at least one movement of the first part which also defines at least one dynamic feature and to provide such stimuli to such a portion of the standard pelvic structure through the movement, where such an actuator member will be referred to as the “actuator member of the second type” throughout this disclosure unless otherwise specified hereinafter. In one example, such a control member may obtain the signal from at least one source, and manipulate the dynamic feature of the movement of the first part based upon (or in at least partial synchronization with) the obtained signal. In another example, the control member may obtain such a signal from at least one source, and manipulate the body and/or actuator members based on (or in at least partial synchronization with) the signal. In another example, the control member may also obtain such multiple signals through at least one source, and then manipulate the actuator member to effect such movement of the first part defining different dynamic features based upon (or in at least partial synchronization with) the signal one at a time (or simultaneously). In another example, such a control member may include a main switch for operating between multiple states each of which represents a different operation of the actuator member, obtain the signal from at least one source based on such a state, and manipulate the dynamic feature of the movement of the first part based upon (or in at least partial synchronization with) the obtained signal. In another example, the control member may obtain the signal from an internet, an external audiovisual device, and/or an external communication device, and manipulate the body and/or actuator members based upon (or in at least partial synchronization with) the signal. In another example, the control member may also include a main switch for moving between multiple states each representing a different operation of the actuator and/or body members, obtain the signal from the states of the main switch, and manipulate the actuator and/or body members based upon (or in at least partial synchronization with) such a signal. In another example, the control member may obtain the signal from at least one source, monitor engagement of such a first part of the body member to the pelvic cavity (or insertion of the first part into the internal cavity), and manipulate the dynamic feature of the movement of the first part based upon (or in at least partial synchronization with) the obtained signal. In another example, the control member may instead obtain the signal from an external audiovisual device, an external communication device, and/or an internet, and manipulate the dynamic feature of the movement of the first part based upon (or in at least partial synchronization with) the obtained signal. In another example, the control member may obtain the signal from a verbal command and/or a bodily command of the user, and manipulate the dynamic feature of the movement of the first part based on (or in at least partial synchronization with) the monitored signal. In another example, the control member may also monitor engagement of the first part with the pelvic cavity (or insertion of the first part in the internal cavity), obtain one of multiple audiovisual (or control) signals based upon an extent and/or a type of the engagement, and then manipulate the actuator and/or body members based upon (or in at least partial synchronization with) the signal. In another example, such a control member may monitor at least one physiological variable of the pelvic structure, obtain such a signal from the variable, and manipulate the dynamic feature of the movement of the first part based on (or in at least partial synchronization with) the monitored signal. In another example, such a control member may monitor at least one physiological variable of the pelvic structure, obtain one of multiple audiovisual (or control) signals based on an extent and/or a type of the engagement, and manipulate the actuator and/or body members based on (or in at least partial synchronization with) the signal.

In another exemplary embodiment of this aspect of the invention, a system may include at least one body member of the first type, at least one actuator member, and at least one control member of the first type. The actuator member may be arranged to effect multiple movements of such a first part each defining at least one dynamic feature for providing the stimuli onto such a portion of the structure through the movements. Such a control member may also obtain the signal from at least one source, select one of the movements of the first part of the body member based on the signal, and manipulate the dynamic feature of the above selected movement of the first part based upon (or in at least partial synchronization with) the signal.

In another exemplary embodiment of this aspect of the invention, a system may include at least one body member of the first type, multiple actuator members, and at least one control member of the first type. Each of the actuator members may be arranged to effect at least one movement of the first part defining at least one dynamic feature for providing the stimuli onto the portion of such a structure through such movement. Such a control member may also obtain the signal from at least one source, select one of the actuator members based upon the signal, and manipulate the dynamic feature of the movement of the first part effected by the selected actuator member based upon (or in at least partial synchronization with) the signal.

In another exemplary embodiment of this aspect of the present invention, a system may include at least one body member, at least one actuator member, and at least one control member of the first type. The body member may be arranged to form multiple first parts each capable of contacting such a portion of the pelvic structure when engaged therewith and to include a second part for providing a grip to an user. The actuator member may be arranged to effect at least one movement of each of the first parts defining at least one dynamic feature for providing the stimuli to the portion of the structure through the movement. The control member may obtain the signal from at least one source, select at least one of the parts based on the signal, and manipulate the dynamic feature of the movement of the selected part based upon (or in at least partial synchronization with) the signal.

In another aspect of the present invention, a pelvic relaxing system may provide tactile stimuli onto at least a portion of the standard pelvic structure based upon at least one audiovisual (or control) signal defining at least one basis.

In one exemplary embodiment of this aspect of such an invention, a system may have at least one body member of the first type, at least one actuator member of the first type, at least one control member of the first type, and the like. In one example, the control member may obtain from at least one source the signal defining a content basis and a voice basis, and manipulate the body and/or actuator members based upon at least one of the bases. In another example, the control member may obtain from at least one source the signal with an action basis but neither a content basis nor a voice basis, and manipulate the body and/or actuator members based on at least one of such bases. In another example, the control member may obtain from at least one source the signal with a background basis but neither a content basis nor a voice basis, and then manipulate the body and/or actuator members based on such an action basis. In another example, the control member may obtain from at least one source the signal defining a content basis and/or voice basis, obtain a background basis, superpose the background basis onto the signal, and manipulate the body and/or actuator members based on the superposed signal. In another example, the control member may obtain from at least one source such a signal defining a background basis, obtain a content basis and/or a voice basis, superpose such a content and/or voice basis onto the signal, and manipulate the body and/or actuator members based upon the superposed signal. In another example, such a control member may obtain from at least one source the signal defining a content basis and/or a voice basis, obtain an action basis, superpose the action basis onto the signal, and then manipulate the body and/or actuator members based upon such a superposed signal. In another example, the control member may obtain from at least one source the signal defining an action basis, obtain a content basis and/or a voice basis, superpose such a voice and/or content basis onto the signal, and manipulate the body and/or actuator members based on the superposed signal. In another example, the control member may obtain from at least one source such a signal defining a background basis, obtain an action basis, superpose such an action basis onto the signal, and manipulate the body and/or actuator members based on the superposed signal. In another example, the control member may obtain from at least one source the signal defining an action basis, obtain a background basis, superpose the background basis to the signal, and manipulate the actuator and/or body members based on the superposed signal.

In another exemplary embodiment of this aspect of the invention, a system may include at least one body member of the first type, at least one actuator member of the first type, at least one control member of the first type, and so on. In one example, the control member may obtain from at least one source multiple audiovisual (or control) signals a first of which may define a content basis whereas a second of which may define a voice basis, synthesize a signal defining the content basis in the voice basis, and manipulate the body and/or actuator members based on the synthesized signal. In another example, the control member may also obtain from at least one source multiple audiovisual (or control) signals a first of which may define a first content basis and a first voice basis of a first person while a second of which may define a second voice basis of a second person, synthesize a signal through replacing the first voice basis by the second voice basis, and manipulate the body and/or actuator members based on the synthesized signal. In another example, the control member may obtain from at least one source multiple audiovisual (or control) signals a first of which may then have a first content basis and a first voice basis of a first person while a second of which may define a second content basis, synthesize a signal through replacing the first content basis by the second content basis, and manipulate the body and/or actuator members based on the synthesized signal. In another example, the control member may obtain from at least one source the signal having a content basis and a voice basis, alter at least a portion of only one of such bases while maintaining the other of the bases, and manipulate the body and/or actuator members based on the altered signal.

In another aspect, a pelvic relaxing system may provide tactile stimuli onto at least a portion of the standard pelvic structure based upon a sound and/or an image.

In one exemplary embodiment of such an aspect of the invention, a system may have at least one body member of the first type, at least one actuator member of the second type, and at least one control member. In one example, the control member may obtain an audiovisual signal for the sound and image based upon the dynamic feature of the movement, and output such a sound and/or image generated from the signal. In another example, the control member may obtain an audiovisual signal for the sound and image based on a verbal command and/or bodily command of the user, and output such a sound and/or image generated from the signal. In another example, the control member may include a main switch for operating between multiple states each representing a different operation of the actuator and/or body members, obtain an audiovisual signal for the sound and image from each of the states of the switch, and output such a sound and/or image generated from the signal. In another example, the control member may monitor engagement of the first part with the pelvic cavity (such as, e.g., insertion of the first part into the internal cavity), obtain one of multiple audiovisual signals for the sound and image based on a type and/or an extent of the engagement, and then output such a sound and/or image generated from the one of the signals. In another example, the control member may also monitor at least one physiological variable of the structure, obtain one of multiple audiovisual signals for the sound and image based on the variable, and output such a sound and/or image from such one of the signals.

In another exemplary embodiment of this aspect of the invention, a system may include at least one body member of the first type, at least one actuator member of the second type, and at least one control member of the first type. In one example, the control member may store multiple pre-recorded audiovisual signals of the user and/or a third party and for the sound and image, obtain at least one of such signals, and manipulate the dynamic feature of the movement based on the obtained signal while outputting the sound and/or image. In another example, the control member may obtain an audiovisual signal of the user for the sound and image, and then manipulate the dynamic feature of the movement based upon the signal while outputting such a sound and/or image. In another example, the control member may obtain at least one pre-recorded audiovisual signal from the user and/or a third party and for the sound and image through an external audiovisual device, and manipulate the dynamic feature of the movement based upon the signal while generating the sound and/or image. In another example, the control member may obtain at least one real-time audiovisual signal of a third party for the sound and image through an external communication device, and then manipulate the dynamic feature of the movement based on the signal while outputting such a sound and/or image. In another example, such a control member may obtain a pre-recorded and/or real-time audiovisual signal of a third party and for the sound and image through an internet, and manipulate the dynamic feature of the movement based on the signal while outputting such a sound and/or image.

In another exemplary embodiment of this aspect of the invention, a system may include at least one body member of the first type, at least one actuator member of the first type, at least one control member of the first type, and the like. In one example, such a control member may obtain at least one audiovisual signal from at least one source, generate the sound and/or image from such a signal, and output the sound while propagating acoustic waves from the sound to the portion, thereby providing the stimuli to the portion not only from the movement of the first part but also by the acoustic waves.

In another exemplary embodiment of this aspect of the invention, a system may include at least one body member of the first type, at least one actuator member of the first type, at least one control member of the first type, and the like. The control member may also output the sound and image as a response to the user input. In one example, the system may include at least one auxiliary driver unit which may be arranged to effect at least one another movement of the first part based on the sound.

In another example, the system may include at least one auxiliary driver unit which may be arranged to effect at least one another movement of the first part based upon the sound and then superpose such another movement of the first part to the movement of the first part effected by the actuator member.

In another exemplary embodiment of the aspect of the invention, a system may include at least one body member, at least one actuator member, and at least one control member of the first type. The body member may be arranged to have multiple first parts each capable of contacting the portion of the pelvic structure when engaged therewith and to include a second part for providing a grip to an user. The actuator member may be arranged to effect at least one movement of at least one of such first parts for providing such stimuli to the portion of the pelvic structure by the movement. The control member may also output the sound and image in response to the user input. The system may include at least one auxiliary driver unit which may be arranged to effect at least one another movement of at least another of the first parts based upon the sound, whereby the system may provide multiple stimuli to multiple the portions not only by such at least one of the first parts but also by at least another of the first parts.

In another aspect of the present invention, a pelvic relaxing system may provide tactile stimuli onto at least a portion of the standard pelvic structure while outputting a sound and/or an image for a preset period of time.

In one exemplary embodiment of this aspect of such an invention, a system may have at least one body member of the first type, at least one actuator member of the first type, at least one control member of the first type, and the like. In one example, such a control member may obtain at least one audiovisual signal based upon the user input (or the dynamic feature of the movement), and output the sound and/or image generated by the signal for the period of time. In another example, such a control member may also obtain a set of multiple audiovisual signals based on the user input (or the dynamic feature of the movement), and output the sound and/or image generated from the set of the signals for the period of time. In another example, the control member may obtain at least two audiovisual signals based upon the user input (or the dynamic feature of the movement), and output such a sound and/or image generated from at least one of such signals for the period of time in a preset (or random) order determined by the user input. In another example, the control member may obtain multiple audiovisual signals based upon the user input (or the dynamic feature of the movement), receive a pre-recorded or real-time control signal from the user and/or a third party, and then output the sound and/or image generated from the audiovisual signals as a response to the control signal for the period of time in a preset order. In another example, the control member may obtain multiple audiovisual signals each of which may define a background basis, an action basis, a voice basis, and/or a content basis based on the user input (or the dynamic feature of the movement), categorize such signals into at least two groups based on at least one of the bases, and output such a sound and/or image generated from the signals which may be selected from a preset group by the user input and/or movement for the period of time. In another example, the control member may obtain multiple audiovisual signals each of which may define a background basis, an action basis, a voice basis, and/or a content basis based on the user input (or the dynamic feature of the movement), categorize such signals into at least two groups based on at least one of such bases, and output the sound and/or image generated from the signals belonging to different groups one at a time in a preset order as determined by such movement and/or user input for the period of time. In another example, the control member may instead obtain multiple audiovisual signals based upon the user input applied to the input and/or sensor units, monitor such a dynamic pattern of the user input applied by a hand of the user and/or pelvic structure, and output the sound and/or image in a preset order for the period of time.

In another exemplary embodiment of this aspect of the invention, a system may include at least one body member of the first type, multiple actuator members, and at least one control member of the first type. Each of such multiple actuator members may be arranged to effect at least one movement of the first part for providing the stimuli onto the portion of the structure through the movement. Such a control member may obtain multiple audiovisual signals based on such an user input (or the dynamic feature of the movement), and output such a sound and/or image generated from the signals based on at least one of the actuator members designated by the user input during the period of time.

In another exemplary embodiment of this aspect of the invention, a system may include at least one body member of the first type, at least one actuator member of the first type, at least one control member of the first type, and the like. The control member may also sense disposition of the first part with respect to the pelvic structure, obtain the signal based on the disposition, and then output such a sound and/or image generated from the signal for the period of time.

In another exemplary embodiment of this aspect of the invention, a system may include at least one body member of the first type, at least one actuator member of the first type, at least one control member of the first type, and so on. In one example, the control member may monitor such a dynamic pattern of the user input, obtain multiple audiovisual signals based on the dynamic pattern, and output such a sound and/or image generated from the signal for the period of time. In another example, the control member may monitor at least one dynamic feature of the movement, obtain multiple audiovisual signals based upon the dynamic feature, and output such a sound and/or image which are generated from the signal for the period of time.

Embodiments of such apparatus aspects of the present invention may include one or more of the following features.

The body member of the first type may include a first unit with at least one first part capable of contacting a single portion (or multiple portions) of the pelvic structure when engaged therewith and include a second unit for providing a grip to an user. The body member may instead include a first unit with multiple first parts all (or at least two but not all) of which may be capable of contacting a single portion (or multiple portions) of such a pelvic structure when engaged therewith and include a second part as well to provide a grip to an user. The control member of the first type may obtain at least one audiovisual (or control) signal from at least one source, and control the actuator member for effecting a single movement (or multiple movements) of a single first part (or multiple parts) while manipulating at least one dynamic feature (or multiple dynamic features) of such movement based upon the obtained signal.

The audiovisual signal may be an analog signal or a digital signal. Such a control member may manipulate the actuator and/or body members based only on an audio signal of the audiovisual signal, only upon a video signal of the audiovisual signal or upon both of the audio and visual signals of such an audiovisual signal. The control member may manipulate the actuator member based upon only one of an audio signal and video signal of the audiovisual signal and may also manipulate the body member based upon only the other of the audio signal and video signal thereof. The control member may also manipulate the actuator and/or body members based upon only one of the content, voice, action, and background bases of the signal, upon only two of the bases, and so on. Such a control member may manipulate the body member based upon one of the content, voice, action, and background bases and may manipulate the actuator member based upon the other of the bases. Such a control member may manipulate the actuator and/or body members without or with the synchronization. In addition, such a control member may also repeat the sound and/or image for the period of time when necessary in the order or in a different order.

The user input may correspond to the audiovisual (or control) signal itself, may correspond to a command signal for selecting the signal from multiple the signals, and the like. The audiovisual signal may include the sound and/or image, may include a command signal for selecting such a sound and/or image from multiple sounds and images, and so on. The system may be arranged to allow the user to reach and to manipulate the input unit (or switch) with at least one finger thereof while simultaneously holding and/or manipulating the second part with a hand thereof during use of such a system without requiring the user to move the hand with respect to the second part. The control member may include conventional on/off switches, speed control switches, and so on. The input unit (or switch) may also receive the user input through at least a substantially identical area thereof for actuating the actuator member, to effect the movements, and to provide the stimuli to the portion. The system may also be arranged to allow the user to provide the user input by contacting and abutting the sensor unit with the portion of the pelvic structure while holding and manipulating the second part with a hand thereof during use of the system without having to move the finger. The input unit (or switch) and/or sensor unit may move or operate between the states at least substantially continuously or intermittently. The input unit (or switch) and/or sensor unit may be a joystick, touch pad, track ball, and other switches which may be arranged to move horizontally and/or vertically while operating between such states. The joystick, touch pad, and/or track ball may also be arranged to move horizontally and/or vertically as a whole in addition to the operating movements.

Such a portion of the pelvic structure may be a clitoris of the entry, a G spot on the wall, other portions of the wall, and the like. Such a part may be defined in its head, trunk, and/or base of such a body member. The body member may include multiple parts which may be identical, disposed close to each other, disposed apart from each other, and the like. The part may also be designed as a clitoral stimulator, as a G spot stimulator, and the like. The dynamic pattern of the user input may include at least one of its temporal pattern and its spatial pattern, where the temporal pattern may include at least one of a duration of the user input, its frequency, and its temporal sequence, and where the spatial pattern may include at least one of an amplitude of the user input (or displacement), its direction, and its type which may be at least one of vibration, horizontal translation, vertical translation, transverse translation, angular rotation, rotation about a center of rotation and/or an axis of rotation defined in the body member, swivelling, tapping, deformation, and the like. The dynamic pattern may be at least one of a frequency of the user input, its temporal rate of change (or acceleration), its displacement (or its integral over time) caused thereby, and a compound value obtained by at least one of mathematical manipulation of at least one thereof. The user input may also be a presence and absence of a contact between the user and input unit (or switch) and/or sensor unit regardless of the force accompanying the contact. The sensor unit may be incorporated into the first part of the body member which may be a head, a trunk, and/or a base thereof. The sensor unit may be incorporated into the second part of the body member.

The dynamic feature of such movement may include at least one of its temporal feature and its spatial feature, where the temporal feature may include at least one of a duration of the movement, its frequency, and its temporal sequence, and where the spatial feature may include at least one of an amplitude of the movement (or displacement), its direction, and its type which may be at least one of vibration, horizontal translation, vertical translation, transverse translation, angular rotation, rotation about a center of rotation and/or an axis of rotation defined in the body member, swivelling, tapping, deformation, and the like. Such a temporal feature may include one or multiple movements of a preset part or different parts of the body member which may be arranged in the sequence which may also be a preset sequence or a sequence which may be at least partially decided by the user input. The spatial feature may include one or multiple movements of a preset part or different parts of the body member while contacting a preset portion or different portions of the pelvic structure.

Such a system may effect multiple different movements of multiple parts, may effect multiple different movements of a single part, may provide different stimuli to a single portion of the structure, and/or may provide the same stimuli to different portions of the structure based upon one or more of such signals without requiring the user to translate, rotate, pivot or otherwise move the system.

In another aspect, a method may provide tactile stimuli effected by at least one part of a body member of a pelvic relaxing system onto at least a portion of such a standard pelvic structure at least partially based upon at least one audiovisual (or control) signal.

In one exemplary embodiment of this aspect of the present invention, a method may include the steps of: obtaining the signal from at least one source; engaging such a body member with the pelvic structure; and manipulating at least one movement of the part of the body member based upon (or in at least partial synchronization with) the signal. Such manipulating may also be replaced by the step of: manipulating a displacement, velocity, and/or acceleration of such a part based on (or in at least partial synchronization with) the signal. Such manipulating may also be replaced by the step of: manipulating a type and/or a direction of the part based upon (or in at least partial synchronization with) the signal. Such manipulating may also be replaced by the step of: manipulating a displacement, velocity, and/or acceleration of at least a substantial part of the system based on (or in at least partial synchronization with) the signal. Such manipulating may be replaced by the step of: manipulating a direction and/or a type of at least a substantial part of the system based upon (or in at least partial synchronization with) the signal.

In another exemplary embodiment of this aspect of the present invention, a method may include the steps of: obtaining the signal from at least one source; engaging the body member with the pelvic structure; and manipulating a displacement, velocity, and/or acceleration of such engaging based on (or in at least partial synchronization with) the signal. Such manipulating may be replaced by the step of: manipulating a type and/or direction of the engaging based on (or in at least partial synchronization with) the signal. Such manipulating may be replaced by the steps of: inserting such a part of the body member into the internal cavity; and manipulating at least one dynamic feature of movement of the part based upon (or in at least partial synchronization with) the signal.

In another exemplary embodiment of this aspect of the present invention, a method may include the steps of: detecting use of the system; obtaining the signal from at least one source upon detecting the use; and manipulating the providing based on (or in at least partial synchronization with) the signal.

In another exemplary embodiment of this aspect of the present invention, a method may include the steps of: providing a main switch capable of turning on and off the system; detecting activation of the switch; obtaining the signal from at least one source upon the detecting; and then manipulating the providing based on (or in at least partial synchronization with) the signal.

In another exemplary embodiment of this aspect of the present invention, a method may include the steps of: providing an actuator member for actuating such a part for effecting the stimuli; detecting activation of the actuator member; obtaining such a signal from at least one source upon the detecting; and manipulating the providing based on (or in at least partial synchronization with) the signal.

In another aspect, a method may be provided for obtaining at least one audiovisual (or control) signal as well as for providing tactile stimuli effected by at least one part of a body member of a pelvic relaxing system onto at least a portion of the standard pelvic structure at least partially based on such a signal.

In one exemplary embodiment of this aspect of the present invention, a method may include the steps of: storing multiple the signals which may be pre-recorded; obtaining at least one signal from the stored signals; engaging such a body member with the pelvic structure; and manipulating at least one movement of the part of the body member based upon (or in at least partial synchronization with) the signal.

In another exemplary embodiment of this aspect of the present invention, a method may include the steps of: obtaining the signal which may be pre-recorded or in real time, may relate to a third party or an user, and may further be provided through an internet, an external audiovisual device, and/or an external communication device; engaging the body member with the pelvic structure; and manipulating at least one movement of the part of the body member based on (or in at least partial synchronization with) the signal.

In another aspect, a method may provide tactile stimuli effected by at least one part of a body member of a pelvic relaxing system onto at least a portion of such a standard pelvic structure while controlling the system through at least one audiovisual (or control) signal.

In one exemplary embodiment of this aspect of the present invention, a method may include the steps of: obtaining the signal from at least one source; engaging such a body member with the pelvic structure; and effecting movement of the part for such stimuli while manipulating at least one dynamic feature of the movement based upon (or in at least partial synchronization with) the signal.

In another exemplary embodiment of this aspect of the present invention, a method may include the steps of: obtaining the signal from at least one of an internet, an external audiovisual device, and an external communication device; engaging the body member with the pelvic structure; and effecting movement of the part for the stimuli while manipulating at least one dynamic feature of the movement based upon (or in at least partial synchronization with) the signal.

In another exemplary embodiment of this aspect of the present invention, a method may include the steps of: providing at least one of a verbal command and a bodily command of the user; obtaining the command as the signal; engaging the body member with the structure; and effecting movement of the part for the stimuli while manipulating at least one dynamic feature of the movement based on (or in at least partial synchronization with) the signal.

In another exemplary embodiment of this aspect of the present invention, a method may include the steps of: arranging the system to effect multiple movements of the part; obtaining the signal from at least one source; selecting one of the movements based on the signal; and then effecting such one of the movements of such a body part. Such selecting and effecting may be replaced by the steps of: selecting at least two of the movements based upon the signal; and effecting such at least two of the movements of the body part in a preset order determined by the signal.

In another exemplary embodiment of this aspect of the present invention, a method may include the steps of: arranging the system to form multiple the parts on the body member; obtaining the signal from at least one source; selecting one of the parts based upon the signal; and effecting at least one movement of the selected body part. Such selecting and effecting may be replaced by the steps of: selecting at least two of the parts based on the signal; and effecting at least one movement of each of the selected body parts one of one at a time and simultaneously as determined by the signal.

In another exemplary embodiment of this aspect of the present invention, a method may include the steps of: arranging the system to define multiple actuator members each of which may be capable of effecting movement of the body part; obtaining the signal from at least one source; selecting one of the actuator members based on the signal; and then effecting at least one movement of the body part by such one of the actuator member. Such selecting and effecting may also be replaced by the steps of: selecting at least two of such actuator members based upon the signal; and effecting at least two movements of the body part through such at least two of the actuator member simultaneously or one at a time as determined by the signal.

In another aspect, a method may provide tactile stimuli effected by at least one part of a body member of a pelvic relaxing system onto at least a portion of such a standard pelvic structure through acoustic waves.

In one exemplary embodiment of this aspect of the present invention, a method may include the steps of: obtaining the signal for at least one of a sound and image; engaging the body member with the pelvic structure; and directing acoustic waves of the sound to the portion of the structure based upon (or in at least partial synchronization with) the signal, thereby providing an additional stimulus to said portion.

In another exemplary embodiment of this aspect of the present invention, a method may include the steps of: providing an auxiliary driver capable of providing an additional stimulus through acoustic waves; obtaining the signal for at least one of a sound and an image; engaging the body member with the pelvic structure; and driving the auxiliary driver with the sound based upon (or in at least partial synchronization with) the signal, thereby providing an additional stimulus to such a portion.

In another exemplary embodiment of this aspect of the present invention, a method may include the steps of: arranging the system to define multiple parts on the body member; providing an auxiliary driver capable of providing an additional stimulus by acoustic waves; obtaining the signal for a sound and/or an image; engaging the body member with the pelvic structure; providing a first stimulus with a first of the body parts in response to the user input; and providing a second acoustic stimulus by the auxiliary driver with the sound based upon (or in at least partial synchronization with) the signal.

In another aspect, a method may provide tactile stimuli effected by at least one part of a body member of a pelvic relaxing system onto at least a portion of such a standard pelvic structure at least partially based upon a synthesized audiovisual (or control) signal.

In one exemplary embodiment of this aspect of the present invention, a method may include the steps of: obtaining a first content basis from a source; obtaining a second voice basis from a different source; defining the first content basis in the second voice basis so as to form the synthesized signal; engaging the body member with the pelvic structure; and manipulating at least one movement of the part of the body member based on (or in at least partial synchronization with) the synthesized signal.

In another exemplary embodiment of this aspect of the present invention, a method may include the steps of: obtaining the signal with a first content basis and a first voice basis; obtaining a second voice basis which is different from the first voice basis; replacing the first voice basis by the second voice basis to from the synthesized signal; engaging the body member with the pelvic structure; and manipulating at least one movement of such a part of the body member based on (or in at least partial synchronization with) the synthesized signal.

In another exemplary embodiment of this aspect of the present invention, a method may include the steps of: obtaining the signal defining a first content basis and a first voice basis; altering the first voice basis to form the synthesized signal; engaging the body member with the pelvic structure; and manipulating at least one movement of such a part of the body member based on (or in at least partial synchronization with) the synthesized signal.

In another aspect, a method may be provided for outputting a sound and/or image from at least one audiovisual (or control) signal and for providing tactile stimuli effected through at least one part of a body member of a pelvic relaxing system to at least a portion of the standard pelvic structure.

In one exemplary embodiment of this aspect of the present invention, a method may include the steps of: engaging the body member with the pelvic structure; effecting at least one movement of the body part; obtaining the signal which is pre-recorded and stored in the system based on (or in at least partial synchronization with) at least one dynamic feature of the movement; and outputting the sound and/or image during the effecting based upon the signal.

In another exemplary embodiment of this aspect of the present invention, a method may include the steps of: engaging the body member with the pelvic structure; effecting at least one movement of the body part; obtaining the signal which may be pre-recorded or in real time and provided by an user and/or a third party based on (or in at least partial synchronization with) at least one dynamic feature of the movement; and outputting the sound and/or image during the effecting based upon the signal.

In another exemplary embodiment of this aspect of the present invention, a method may include the steps of: engaging the body member with the pelvic structure; effecting at least one movement of the body part; obtaining the signal which may be pre-recorded or in real time and provided through an external audiovisual device, an external communication device, and/or internet based on (or in at least partial synchronization with) at least one dynamic feature of the movement; and outputting the sound and/or image during the effecting based upon the signal.

In another exemplary embodiment of this aspect of the present invention, a method may include the steps of: engaging the body member with the pelvic structure; effecting at least one movement of the body part; obtaining at least two of such signals; combining the signals to form a compound signal; and outputting the sound and/or image during such effecting at least partially based on the compound signal.

In another exemplary embodiment of this aspect of the present invention, a method may include the steps of: engaging the body member with the pelvic structure; effecting at least one movement of the body part; obtaining at least two of such signals; synthesizing such signals to form a synthesized signal; and outputting such a sound and/or image during such effecting at least partially based on the synthesized signal.

In another aspect, a method may be provided for providing tactile stimuli effected by at least a part of a body member of a pelvic relaxing system to at least a portion of the standard pelvic structure while outputting a sound and/or an image for a preset period of time.

In one exemplary embodiment of this aspect of the present invention, a method may include the steps of: engaging the body member with the pelvic structure; effecting at least one movement of the body part; obtaining at least one audiovisual signal for the sound and/or image; and then outputting the sound and/or image based upon (or in at least partial synchronization with) the signal for the period of time while repeating the outputting when necessary. Such obtaining and outputting may be replaced by the steps of: obtaining multiple audiovisual signals for the sound and/or image; categorizing such signals into multiple groups; and outputting the sound and/or image of one of such groups selected by as well as based on (or in at least partial synchronization with) the signal for the period of time while repeating the outputting when necessary. Such obtaining and outputting may also be replaced by the steps of: obtaining multiple audiovisual signals for at least one of the sound and image; and outputting such multiple sounds and/or images in a preset order selected by and based upon (or in at least partial synchronization with) the signal for the period of time while repeating the outputting when necessary. Such obtaining and outputting may be replaced by the steps of: obtaining multiple audiovisual signals for the sound and/or image; receiving an external control signal for selecting a preset order through an external audio device, an external communication device, and/or an internet; and outputting multiple sounds and/or images in the order for the period of time while repeating the outputting as necessary.

In another exemplary embodiment of this aspect of the present invention, a method may include the steps of: engaging the body member with the structure; effecting at least one movement of such a body part; obtaining multiple audiovisual signals for the sound and/or image; generating a synthesized signal from the signals; and then outputting such multiple sounds and/or images based upon (or in at least partial synchronization with) the synthesized signal for the period of time while repeating such outputting when necessary.

Embodiments of the above method aspects of the present invention may include one or more of the following features.

The providing the stimuli may include at least one of the steps of: providing mechanical friction onto the portion; providing mechanical impact onto the portion; moving and/or pushing the portion with respect too other portions of the pelvic structure; providing sustained contact with such a portion, and the like. The obtaining such a signal may include at least one of the steps of: acquiring the signal in an analog format; acquiring the signal in a digital format; acquiring an analog signal followed by digitizing the signal into a digital signal, and the like. The obtaining such a signal may include at least one of the steps of: acquiring the audiovisual signal for the sound and/or image; and acquiring the control signal capable of rendering the control member generate the audiovisual signal. The engaging may include the step of: contacting the part with a clitoris of the entry, a G spot on the wall, other portions of the wall, and the like.

Such effecting and/or manipulating may include at least one of the steps of: effecting different movements defining different dynamic features; and changing at least one configuration of the body member which may be at least one of a length of the body member, its diameter, its curvature, and its surface texture. The effecting and/or manipulating may include at least one of the steps of: controlling temporal feature and/or spatial feature of such movement; controlling at least one of a duration of the movement, its frequency, and its temporal sequence; controlling at least one of an amplitude of such movement (or displacement), its direction, and its type; and selecting the movement from at least one of horizontal, vertical or transverse translation, vibration, angular rotation, rotation about a center of rotation or an axis of rotation defined in the body member, swivelling, tapping, deformation, and the like. The effecting and/or manipulating may include the step of: effecting one or multiple movements of a preset part (or different parts) of the body member which may be arranged in the sequence which may in turn be a preset sequence or a sequence which may be at least partially determined by the user input. The effecting and/or manipulating may further include the step of: effecting one or multiple movements of a preset part (or different parts) of the body member and contacting a preset portion (or different portions) of the pelvic structure.

The storing may include at least one of the steps of: storing the audiovisual signals without or with compression; and storing the control signals without or with compression. Such selecting may include at least one of the steps of: selecting one of the parts, movements, and actuator members by the user input which is supplied to the system by a hand of an user; and selecting one of the parts, movements, and actuator members by the user input supplied thereto through the pelvic structure of the user. The outputting may include at least one of the steps of: playing the sound through a speaker disposed in the body member, playing the sound with a speaker of an external audiovisual device; playing the sound by a speaker of an external communication device; displaying the image through a screen disposed in the body member; displaying the image with a screen of an external audiovisual device; and displaying the image by a screen of an external communication device. The categorizing may include at least one of the steps of: grouping the signals based upon at least one of the content, voice, and action bases; grouping the signals based upon lengths thereof; grouping the signals based upon a gender providing the signals, and the like. The combining may include at least one of the steps of: appending one of the signals on a beginning or an end of another of the signals; superposing one of the signals onto another of the signals; performing the superposing proceeded by (or followed by) at least one filtering of the signals.

The method may further include one of the steps of: effecting multiple different movements of multiple parts of the first unit; effecting multiple different movements of a single part of the first unit; providing different stimuli to a single portion of the structure with a single or multiple parts of the first unit; and providing the same stimuli to different portions of the structure with a single or multiple parts of the first unit, where all of such effectings may be based on one or more of such audiovisual and/or control signals without requiring the user to translate, rotate, pivot or otherwise move the system.

In another aspect of the present invention, a pelvic relaxing system may provide tactile stimuli to at least a portion of the standard pelvic structure at least partially based on at least one audiovisual (or control) signal.

In one exemplary embodiment of this aspect of the invention, a system may be provided by a process including the steps of: defining at least one first part and at least one second part in at least one body member, where the first part may contact the portion of the pelvic structure when engaged therewith and where the second part may provide a grip to an user; arranging at least one actuator member to effect at least one movement of the first part for providing the stimuli onto the portion of the structure through the movement; and providing a control member with at least one of at least one input unit and at least one sensor unit for receiving an user input with at least one dynamic pattern thereby; effecting the movement of the first part based on the dynamic pattern of the user input; obtaining the signal by the control member; and manipulating the body and/or actuator members through the control member based on the signal during use of the system. Such obtaining and manipulating may then be replaced by the steps of: obtaining at least one signal from such multiple signals through the control member based upon the user input; and manipulating the body and/or actuator members by the control member based on the obtained signal. Such obtaining and manipulating may be replaced by the steps of: incorporating a main switch capable of operating between multiple states each defining a different operation of the actuator member; obtaining at least one signal from such multiple signals through the control member based on the state of the main switch; and then manipulating the body and/or actuator members with the control member based upon the obtained signal. Such obtaining and manipulating may be replaced by the steps of: obtaining at least one signal from multiple the signals with the control member based on insertion (or an extent of insertion) of the first unit into the internal cavity; and then manipulating the body and/or actuator members with the control member based on the obtained signal. Such obtaining and manipulating may be replaced by the steps of: obtaining at least one signal from such multiple signals with the control member based on another movement of the system as a whole; and manipulating the body and/or actuator members with the control member based upon the obtained signal.

In another exemplary embodiment of this aspect of the invention, a system may be provided by a process including the steps of: the above defining, arranging, providing, and effecting in this order; providing the control member with at least one storage unit for storing multiple the signals; obtaining at least one signal from the storage unit; and manipulating the body and/or actuator members through the control member based upon (or in at least partial synchronization with) such a retrieved signal. Such providing, obtaining, and manipulating may be replaced by the steps of: obtaining at least one of such signals from an external audiovisual device; and manipulating the actuator and/or body members with the control member based upon (or in at least partial synchronization with) the obtained signal. Such providing, obtaining, and manipulating may be replaced by the steps of: obtaining at least one of the signals from an external communication device; and manipulating the body and/or actuator members with the control member based upon (or in at least partial synchronization with) the obtained signal. Such providing, obtaining, and manipulating may be replaced by the steps of: obtaining at least one of the signals through an internet; and manipulating the body and/or actuator members with the control member based upon (or in at least partial synchronization with) the obtained signal. Such providing, obtaining, and manipulating may also be replaced by the steps of: obtaining at least two of the signals; combining the signals by the control member to form a compound signal; and manipulating the actuator and/or body members with the control member based upon (or in at least partial synchronization with) the compound signal. Such providing, obtaining, and manipulating may also be replaced by the steps of: obtaining a first signal defining a first content basis and a first voice basis from multiple the signals; obtaining a second signal having a second content basis and a second voice basis from such multiple signals; replacing one of the bases of one of the first and second signals by such one of the bases of another of the first and second signals by the control member to form a synthesized signal; and then manipulating the actuator and/or body members with the control member based on (or in at least partial synchronization with) the synthesized signal. Such providing, obtaining, and manipulating may also be replaced by the steps of: obtaining a first signal defining a preset content basis and voice basis from multiple the signals; altering at least one of the bases of the first signal with the control member; and manipulating the actuator and/or body members with the control member based on (or in at least partial synchronization with) the altered signal.

More product-by-process claims may be constructed by modifying the foregoing preambles or their modifications of the above system and/or method claims and by appending thereto the bodies or their modifications of the above system and/or method claims. Such product-by-process claims may be arranged to include one or more of the above features of such system and/or method claims of the present invention.

As used herein, both of the terms “input unit” and “sensor unit” refer to those units of various pelvic relaxing systems capable of receiving user inputs and/or monitoring dynamic pattern thereof. However, the “input unit” and “sensor unit” are to be differentiated as follows within the scope of the present invention. First of all, the “input unit” is generally disposed in a part of a body member of such a system which is intended to not be inserted into a cavity of a pelvic structure, whereas the “sensor unit” is preferably disposed in such an insertable part of the body member. Therefore, the “input unit” mainly receives the user inputs through a hand or finger of an user, while the “sensor unit” generally receives the user inputs through various portions of the pelvic structure.

The terms “proximal” and “distal” will be used in a relative context. Throughout this invention, the term “proximal” is to be used to denote a direction toward a head of a body member of a system, while the term “distal” is to be used to denote an opposite direction toward an end of a handle of such a system. Accordingly, a “proximal” end and a “distal” end may be defined with respect to an entire pelvic relaxing system or with respect to a specific member or unit thereof.

The terms “input unit” and “sensor unit” generally refer to identical or similar articles capable of monitoring various dynamic patterns of various user inputs applied thereto. Throughout this invention, however, the “input units” represent such articles incorporated into a handle part (or a second unit) of a body member of a pelvic relaxing system, while the “sensor units” denote such articles incorporated into an insertable part (or a first unit) of such a body member. Accordingly, any articles which may be used as the “input unit” may also be used as the “sensor unit” unless otherwise specified. In addition, the “input unit” may also be disposed in the insertable part of the body member, while the “sensor unit” may also be disposed in the handle part thereof when desired.

As used herein, a “dynamic pattern” refers to a temporal pattern as well as a spatial pattern of an user input and/or of a sensing signal each of which is generated by an input unit and/or a sensor unit of a control member in response to the user input, while a “dynamic feature” refers to a temporal feature as well as a spatial feature of movement of a single part or multiple parts of a body member.

A “pelvic structure” means an anatomical structure including an entry and a wall, where such an entry forms an orifice therethrough, while the wall includes muscles and defines an internal cavity extending inwardly from the entry and bound by the muscles. Such a pelvic structure will be referred to as the “standard pelvic structure” or simply the “pelvic structure” hereinafter for ease of illustration, unless otherwise specified.

Unless otherwise defined in the following specification, all technical and scientific terms used herein have the same meaning as commonly understood by one of ordinary skill in the art to which the present invention belongs. Although the methods or materials equivalent or similar to those described herein can be used in the practice or in the testing of the present invention, the suitable methods and materials are described below. All publications, patent applications, patents, and/or other references mentioned herein (particularly those enumerated in the above Background section) are incorporated by reference in their entirety. In case of any conflict, the present specification, including definitions, will control. In addition, the materials, methods, and examples are illustrative only and not intended to be limiting.

Other features and advantages of the present invention will be apparent from the following detailed description, and from the claims.

### BRIEF DESCRIPTION OF THE DRAWING

FIGS. 1A to 1F are schematic views of exemplary pelvic relaxing systems capable of obtaining audiovisual or control signals and synchronizing various relaxing operations thereof with such signals according to the present invention; and

FIGS. 2A and 2B represent schematic diagrams of various functional members and units of an exemplary pelvic relaxing system according to the present invention.

### DETAILED DESCRIPTION OF THE PREFERRED EMBODIMENTS

The present invention generally relates to pelvic relaxing systems capable of obtaining various audiovisual (or control) signals and synchronizing various relaxing operations of such systems with such signals. More particularly, the present invention relates to various pelvic relaxing systems which obtain pre-recorded and/or real-time audiovisual (or control) signals from an user and/or a third party, analyze various bases carried by such signals, and synchronize various operations of such systems with one or more of such bases. Therefore, such pelvic relaxing systems may provide various stimuli onto one or multiple portions of the pelvic structure in temporal and/or spatial synchronization with the signals. The systems may also preferably effect movements of various parts thereof, effect various movements of a single part or multiple parts thereof, and/or provide such stimuli to various portions of the pelvic structure without requiring an user to reciprocate, rotate, and/or otherwise move the entire system. Accordingly, the user may receive such stimuli which are synchronized with various bases carried by such signals without having to manually imitate the sounds and/or images of such signals. Conversely, the present invention also relates to various pelvic relaxing systems which may generate audiovisual signals which may be synchronized with various operations of the systems. Accordingly, the user may listen to the sounds and/or watch the images by audiovisual output units during manual operation of the system such as, e.g., movements of such parts of the system and/or entire system, various features of such movements, and the like.

The present invention also relates to various methods of obtaining the audiovisual (or control) signals from various sources, obtaining such compound and/or synchronized signals by manipulating such audiovisual (or control) signals, and synchronizing various operations of the system with such signals. Conversely, the present invention relates to various methods of effecting a single movement or multiple movements of one or multiple parts the system by one or multiple actuator members thereof, alternatively generating manual movements of an entire system, and generating sounds and/or images synchronized with one or more of such movements. The present invention further relates to various processes for providing the above system and various members, units, and/or parts thereof capable of obtaining the signals and synchronizing various operations of such a system with the signals. The present invention also relates various processes for providing such a system and various members, units, and/or parts thereof capable of generating the sounds and/or images synchronized with one or more of such movements of the parts of the system and/or those of the entire system.

Various aspects and/or embodiments of various systems, methods, and/or processes of this invention will now be described more particularly with reference to the accompanying drawings and text, where such aspects and/or embodiments thereof only represent different forms. Such systems, methods, and/or processes of this invention, however, may also be embodied in many other different forms and, accordingly, should not be limited to such aspects and/or embodiments which are set forth herein. Rather, various exemplary aspects and/or embodiments described herein are provided so that this disclosure will be thorough and complete, and fully convey the scope of the present invention to one of ordinary skill in the relevant art.

Unless otherwise specified, it is to be understood that various members, units, elements, and parts of various systems of the present invention are not typically drawn to scales and/or proportions for ease of illustration. It is also to be understood that such members, units, elements, and/or parts of various systems of this invention designated by the same numerals may typically represent the same, similar, and/or functionally equivalent members, units, elements, and/or parts thereof, respectively.

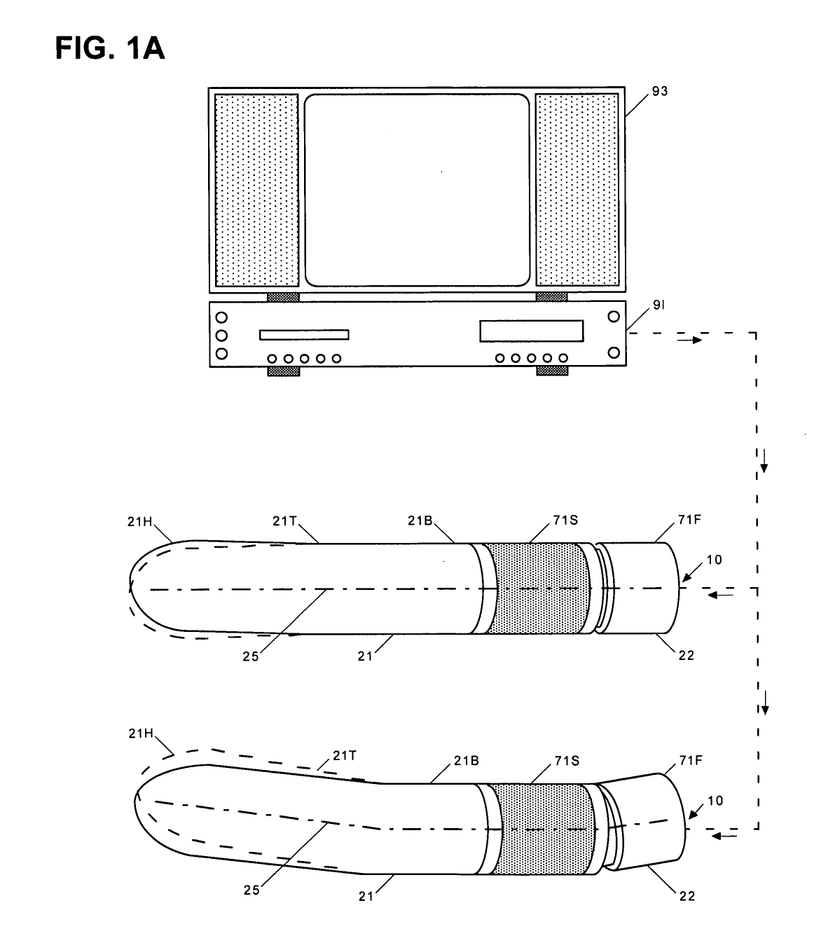

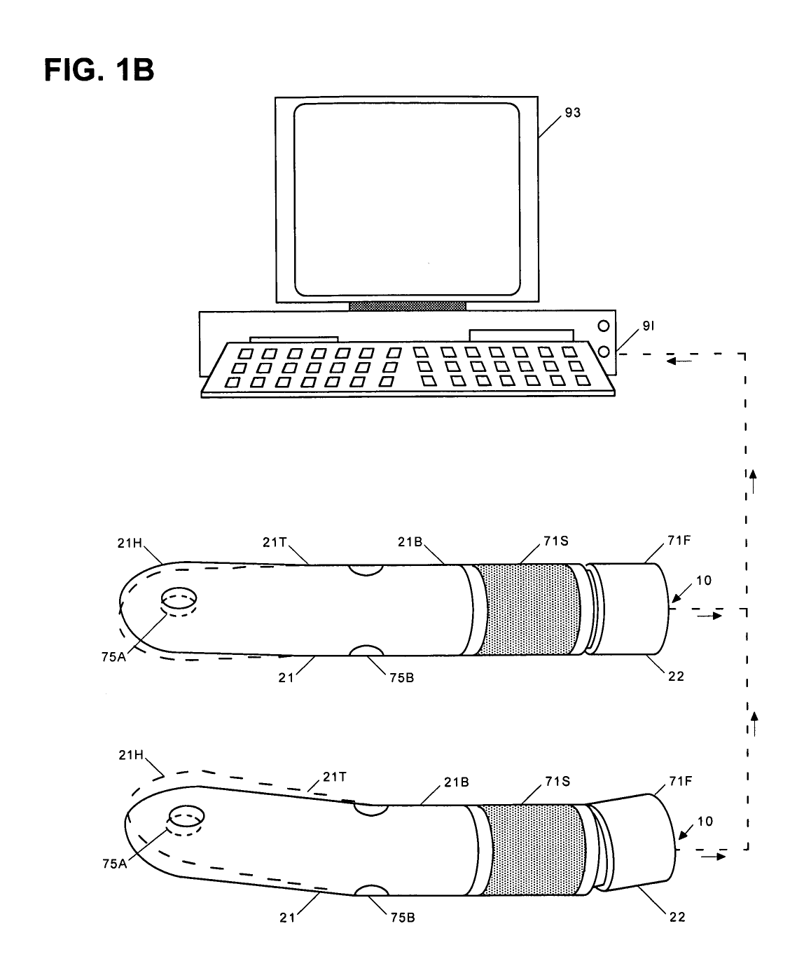

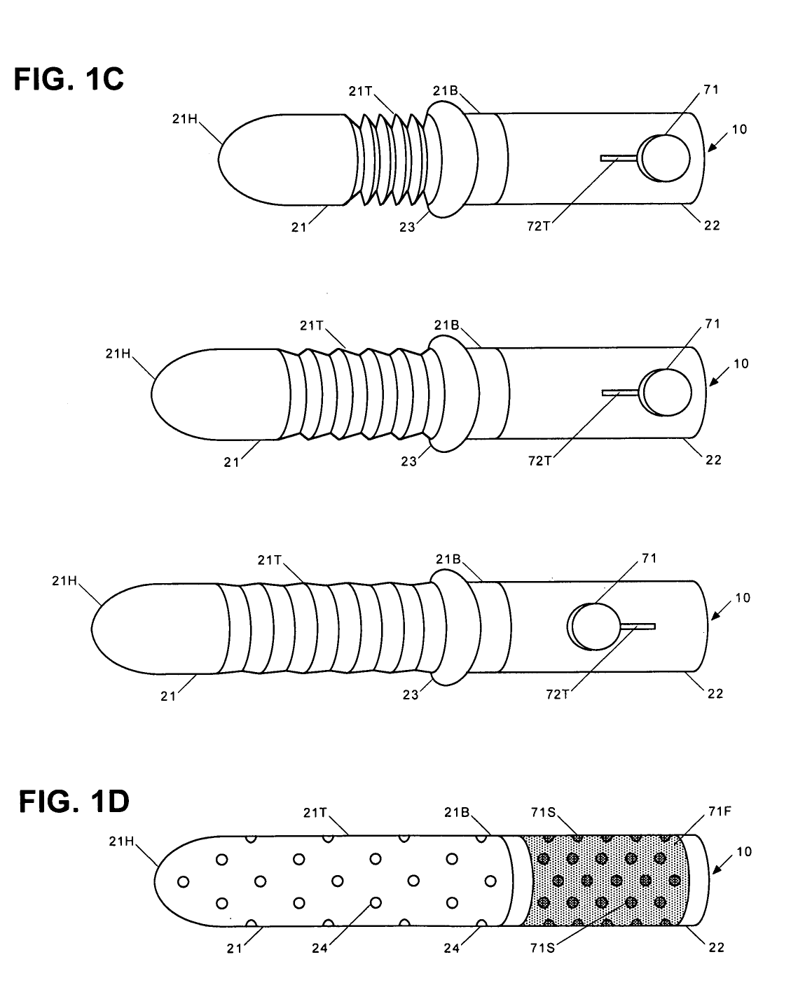

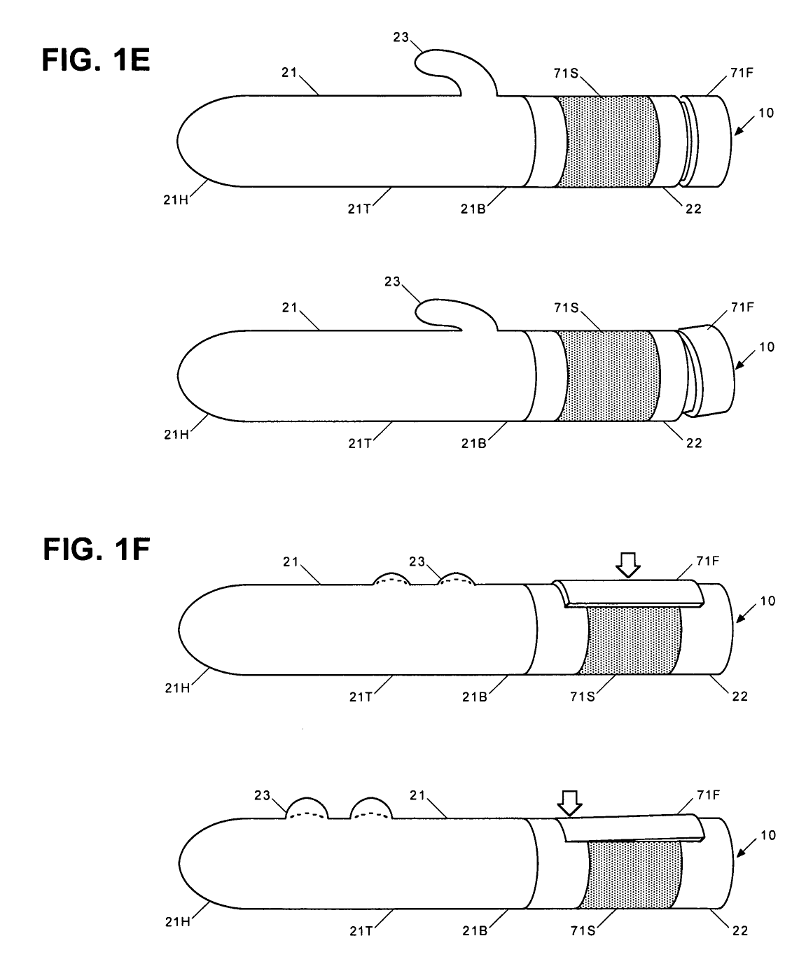

In one aspect of the present invention, pelvic relaxing systems may be provided with various members and units for effecting various movements and providing various stimuli to a portion and/or multiple portions of such a structure while synchronizing such movements and/or stimuli with various audiovisual and/or control signals supplied thereto by various internal and/or external sources. FIGS. 1A to 1E are schematic views of exemplary pelvic relaxing systems capable of obtaining audiovisual or control signals and synchronizing various relaxing operations thereof with such signals according to the present invention.

In one exemplary embodiment of this aspect of the present invention and as described in FIG. 1A, an exemplary pelvic relaxing system 10 operatively couples with at least one external audiovisual output unit 93 which is in turn operatively coupled to at least one audiovisual input unit 91. The system 10 typically includes a body member 20 with a first unit 21 and a second unit 22, an actuator member incorporated inside such first and second units 21, 22 of the body member 20 (thus not shown in this figure), and a control member including a first input unit 71F, a second input unit 71B, and other units which may be incorporated inside the body member 20 (thus not shown in this figure). As described in the middle panel and bottom panel of FIG. 1A, at least a portion of the first unit 21 is elongated and also arranged to be inserted into an internal cavity of a pelvic structure during use of the system 10, while the second unit 22 is distally coupled to the first unit 21 and arranged to form a grip for an user during use. The first unit 21 is divided into a head 21H, a trunk 21T, and a base 21B. The head 21H is formed in a proximal end of the body member 20 and preferably rounded in order to facilitate insertion of the body member 20 into the internal cavity. The trunk 21T is defined distal to such a head 21H and terminates in the base 21B which then couples to the second unit 22. In general, the head 21H, trunk 21T, and base 21B are fixedly or movably coupled to each other, where the base 21B may be coupled to the second unit 22 fixedly or releasably. In the alternative, the head 21H, trunk 21T, and base 21B may be arranged to form an unitary first unit 21 which then couples with the second unit 72 fixedly or releasably. As far as the body member 20 may generate one or more desirable movements of at least one part thereof, detailed construction and coupling modes of such parts 21H, 21T, 21B are generally not material to the scope of the present invention. The second unit 22 is also elongated and defines a shape and/or size to form the grip for the user, whether the user grabs the second unit 22 with his or her thumb pointing proximally, distally or laterally. The first and second units 21, 22 may be movably or fixedly coupled to each other and form an unitary body member 20. Alternatively, the first and second units 21, 22 may define an unitary body member 20. As far as the body member 20 may generate one or more desirable movements, detailed construction and/or coupling modes of such first and second units 21, 22 are neither material to the scope of the present invention. As will be described below, the pelvic relaxing system 10 also includes other members and units for normal operation. When the first and/or second units 21, 22 may form cavities therein, such members and/or units may be incorporated into one or both of such units 21, 22. Alternatively, at least one of such members and/or units may be exposed through surfaces of such units 21, 22.

As depicted in the top panel of FIG. 1A, at least one external audiovisual input unit 91 (or to be abbreviated as an external “A/V input unit” hereinafter) may generate or obtain various audiovisual (or control) signals and then supply such signals to the system 10, e.g., to an audiovisual input unit of the control member (not included in the figure) of the system 10. Such an external A/V input unit 91 may then optionally supply such audiovisual (or control) signals to at least one external audiovisual output unit 93 (or to be abbreviated as an external “A/V output unit” hereinafter) which then generate various sounds and/or images as represented or carried by such signals. In the embodiment of FIG. 1A, such an external A/V input unit 91 is a conventional VCR, CD or DVD player, while the external A/V output unit is a conventional TV or monitor hooked to the external A/V input unit 91. When desirable, such an external output unit 93 may also generate or obtain the audiovisual (or control) signals and then supply such signals to the audiovisual input unit of the system 10 which is to be abbreviated as an “internal A/V unit”) hereinafter, where the external A/V input unit 91 may not be required for various relaxing operations of the system 10. Regardless of their sources, such audiovisual (or control) signals may be generated by or related to the user of the system 10 or a third party, may be pre-recorded signals or real-time signals. In addition, such audiovisual (or control) signals may be analog or digital signals.

The first input unit 71F is movably incorporated onto a distal end of the second unit 22 and also oriented so that an user may apply an user input thereto through at least an area of the first input unit 71F vertically, horizontally, at an angle or angularly. Similar to conventional joysticks, such a first input unit 71F moves or operates between multiple states, and responds to various dynamic patterns of the user input such as, e.g., movement of a portion or an entire portion of the first input unit 71F effected by the user, temporal patterns of such movement, and spatial patterns thereof. The first input unit 71F generates sensing signals in response to the dynamic patterns and delivers the signals to the control member which may manipulate other members and/or units of the system 10 in order to effect various movements of a single part or multiple parts of the first input unit 21F or, in the alternative, to change at least one configuration of the body member 20, where examples of such configurations may include, but not be limited to, a length of the first input unit 21, its width, its height or diameter, its curvature, its surface texture, and the like. The first input unit 71F is also shaped and/or sized in order to allow the user to manipulate the first input unit 71F while grabbing or holding such a second unit 22 whether the user grabs the second unit 22 with the thumb pointing proximally, distally, laterally, and the like. More specifically, the first input unit 71F may be arranged to changes the configuration of the first unit 21 or to generate sensing signals of which amplitudes, directions, durations or other temporal and/or spatial patterns may be determined by various dynamic patterns of the user input for grabbing and/or holding the second unit 22. Therefore, the user may control various relaxing operations of such a system 10 while grabbing and holding such a second unit 22 during use and without requiring the user to change the grip of the second unit 22 and/or to move the hand for the purpose of reaching the first input unit 71F while holding or grabbing the second unit 22, and the like.

Contrary to the first input unit 71F incorporated into the distal end of the second unit 22 of the body member 20, the second input unit 71S is exposed around the second unit 22 for providing direct access to the user. Such a second input unit 71S of this embodiment is arranged to receive the user input through at least a substantial area thereof. The second input unit 71S is also incorporated into a center part of the second unit 22, while forming vacant areas between itself 71S and the proximal and distal ends of the second unit 22. Therefore, the second input unit 71S shares an area of the second unit 22 which is also used to define the grip for the user. As will be explained in greater detail below, the second input unit 71S is preferably arranged to monitor various dynamic patterns of mechanical or electrical contact with the user and/or to monitor those of force applied thereto by the user, and then to generate sensing signals based on such dynamic patterns. More specifically, the second input unit 71S may also generate the sensing signals of which amplitudes, directions, durations or other spatial and/or temporal patterns may be determined by the contact and/or force for grabbing and holding the second unit 22. Accordingly, the user may control various relaxing operations of the system 10 while grabbing and holding the second unit 22 during use and without requiring the user to change the grip of the second unit 22 and/or to move the hand for reaching the second input unit 71S while holding or grabbing the second unit 22, and the like. Further configurational and/or operational characteristics of such a second input unit 71S may be similar or identical to those of various input units of the variable resistance types described in the co-pending Applications.

In operation, the first and second units 21, 22 are fixedly or movably coupled with each other to form the body member 20, and the system 10 is connected to an electric power outlet with a power supply cable (not shown in the figure) or provided with a battery. The user then inserts the head 21H of the first unit 21 into the internal cavity. When the user pivots, swivels or otherwise manipulates the first input unit 71F and moves such an unit 71F to each of its states, the first input unit 71F generates the sensing signals based on one or more dynamic patterns of the user input and delivers the sensing signals to the control member (not included in the figure). Based upon dynamic patterns of such user inputs, the control member manipulates the actuator member to effect rotation of the first unit 21 about the longitudinal axis 25 of the body member 20 along the circular path, thereby providing stimuli to the circular portion of such a pelvic structure. When the user desires different movements of the rotating first unit 21, she may apply different user inputs by simply pushing or swivelling the first input unit 71F along a preset direction without having to move her hands along or around the second unit 22 and/or without having to change the grip onto the second unit 22. The first input unit 71F generates different sensing signals in response to, based upon or in synchronization with the user inputs and sends the signals to the control member which then manipulates the actuator member to change the curvature of the first unit 21, e.g., by increasing the acute angle (or decreasing the obtuse angle) formed between the straight and curved parts of the body member 20 and increasing a radius of circular movement of the first unit 21 as described in the bottom panel of FIG. 1A or vice versa. Alternatively, the first input unit 71F may be mechanically coupled to the first unit 21 so that movement of the first input unit 71F in one direction may bend the first unit 21 along the same (or opposite) direction. When the user desires to change a speed or intensity of vibration of the first unit 21, she may change the dynamic pattern of the user inputs applied to the second input unit 71S, e.g., by squeezing the second input unit 71S, by contacting such an unit 71S for a longer period of time, and the like. In response to, based upon or in synchronization with such user inputs, the second input unit 71S generates different sensing signals and delivers such signals to the control member which may manipulate the actuator member to rotate in different speeds. Accordingly, the user may effect multiple movements of the body member 20 and provide different stimuli based upon, in response to or in synchronization with multiple user inputs.

Still referring to the middle and bottom panels of FIG. 1A, the actuator member may be arranged to effect one or multiple movements of a single part or multiple parts of the first unit 21 or may change at least one configuration of the first unit 21 for generating stimuli and delivering the stimuli to a portion of the pelvic structure which is contacted or abutted by the part of the first unit 21. In the embodiment of FIG. 1A, the body member 20 defines a longitudinal axis 25 which is typically straight from the distal end of the second unit 22 to the trunk 21T of the first unit 21 and then curved at an acute angle to the proximal end of the first unit 21. In addition, the actuator member is arranged to rotate the first unit 21 about the straight part of the longitudinal axis 25 so that the distal part of the first unit 21 rotates along a circular path which is normal or perpendicular to the longitudinal axis 25 as depicted by the solid and dotted lines shown in the middle panel. The second input unit 71S may then be arranged to manipulate a speed of rotation of the first unit 21 around the longitudinal axis 25 depending upon various dynamic patterns of the user input. In addition, the first input unit 71F may also be arranged to manipulate such an angle defined between the straight and curved parts of the longitudinal axis 25 so that moving the first input unit 71F along one direction increases the angle, whereas moving such a first input unit 71F along an opposite direction decreases the angle. Accordingly and as depicted in the bottom panel of FIG. 1A, pivoting or swivelling the first input unit 71F upwardly causes the curved proximal part of the first unit 21 to bend upwardly as well and to rotate along another circular path which is also normal or perpendicular to the curved longitudinal axis 25.

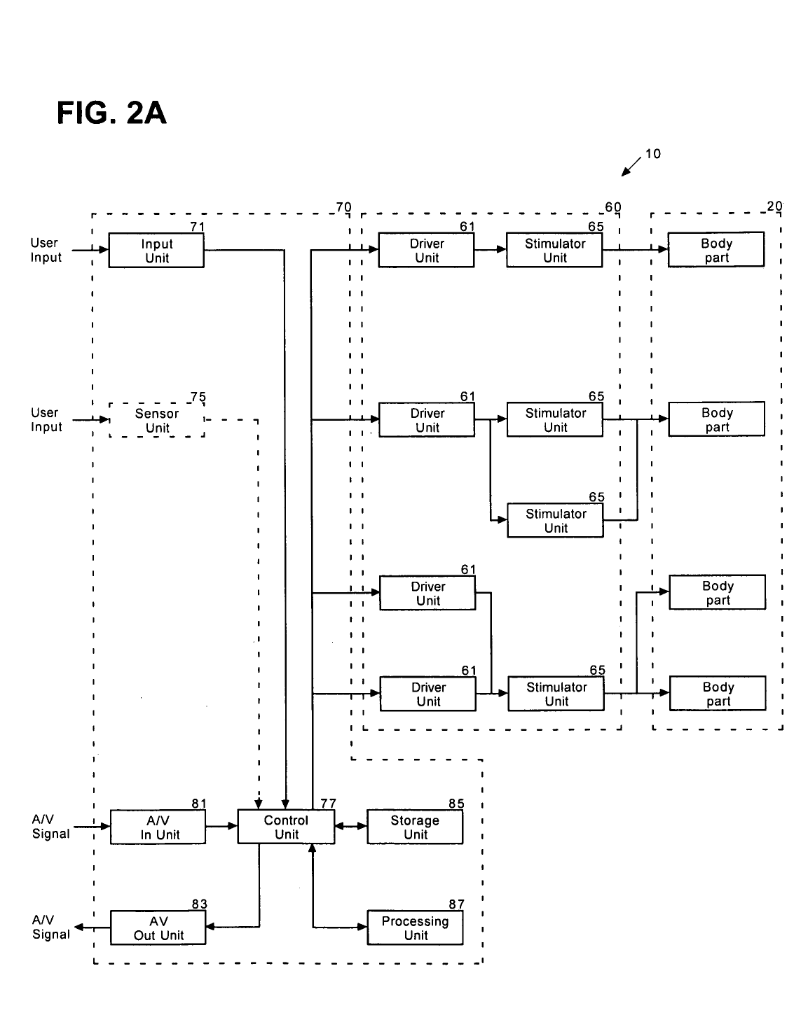

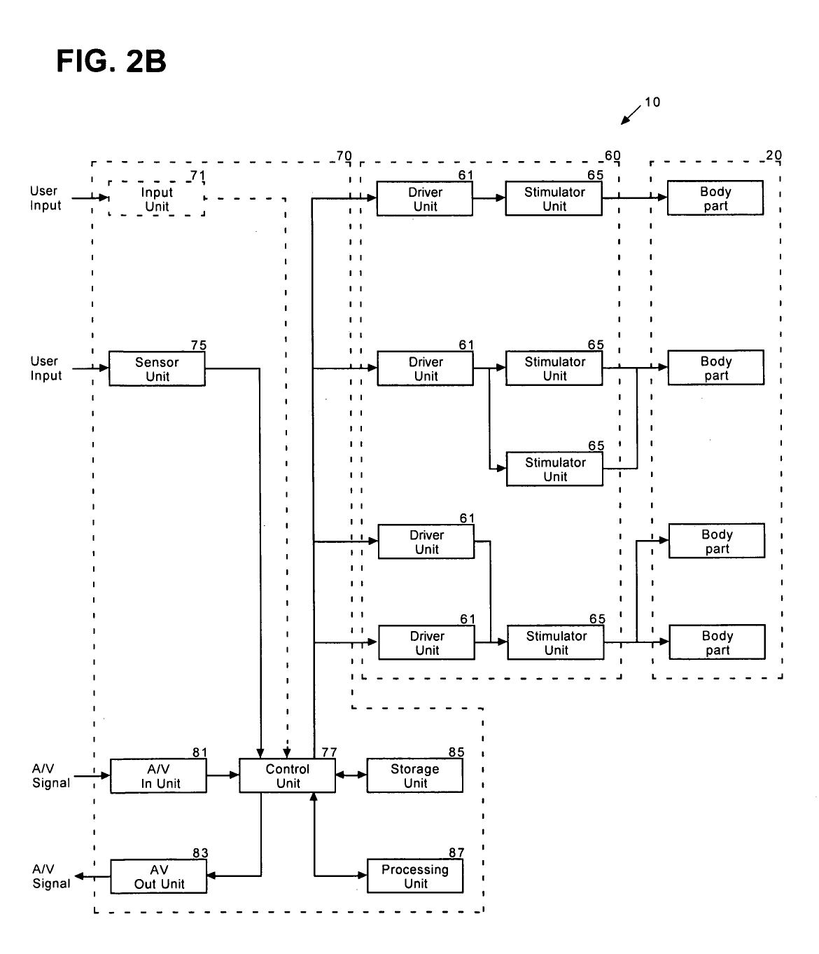

The control member may also have at least one audiovisual input unit (or to be abbreviated as an “internal A/V input unit” hereinafter) and at least one audiovisual output unit (or to be abbreviated as an “internal A/V output unit hereinafter), where the main function of the internal A/V input unit is to generate or obtain the audiovisual (or control) signals from various sources, while the main function of the internal A/V output unit is to generate various images and/or sounds based upon the generated or obtained audiovisual (or control) signals. Although not shown in the figure, the control member may include at least one storage unit which may receive and store various audiovisual (or control) signals. The storage unit may be fabricated of any conventional data storage articles such as, e.g., magnetic tapes, magnetic disks, optical disks, microchips, and other data storage devices for temporarily and/or permanently storing analog or digital data therein. Depending upon types of such articles, the control member may include suitable drivers to operate such a storage unit, where examples of such drivers may include, but not be limited to, magnetic or optical tape drivers, magnetic or optical disk drivers, and so on. The storage unit may be disposed in various parts of the body member 20 and, therefore, may be disposed inside or on the first and/or second units 21, 22. Further configurational details of such a control member are to be provided in conjunction with FIGS. 2A and 2B. Other configurational and/or operational characteristics of the system of FIG. 1A are similar or identical to various systems which have been disclosed in the co-pending Applications.

Still referring to FIG. 1A but in another exemplary embodiment of such an aspect of the present invention, an exemplary pelvic relaxing system 10 may be operatively coupled to at least one external A/V input unit 91 and/or at least one external A/V output unit 93 and may effect various movements of a single or multiple parts of its body member 20 and/or configurational changes of such a member 20. The body member 20 of such a system 10 is identical or similar to that of the previous embodiment and the actuator member of the system 10 is also arranged to effect identical or similar movements of the body member 20 and/or its configurational changes. In contrary to the manual system control through the first and second input units 71F, 71S as described in the previous embodiment, the system 10 may receive such audiovisual (or control) signals through the external A/V input and/or output units 91, 93 and may effect the movements and/or configurational changes based thereupon, in response thereto, and/or in synchronization therewith, i.e., this embodiment relies upon control of the system 10 through various signals (and to be referred to as a “signal system control” in contrary to the foregoing “manual system control”). In general, the audiovisual signals carry content bases, voice bases, action bases, and/or background bases, while the control member may be arranged to effect the movements and/or configurational changes based on, in response to, and/or in synchronization with at least one of such bases. In the alternative, the control signals may be arranged to carry pulse trains of electric voltage or current, and the control member may effect such movements and/or configurational changes based upon, in response to or in synchronization with the pulse trains thereof.

It is to be understood that a main difference between the “manual system control mode” of the previous embodiment and the “signal system control mode” of this embodiment lies in the facts that the “manual system control” relies upon the user inputs supplied to the system 10 through its input units, but that the “signal system control” depends upon the audiovisual (or control) signals supplied to the system 10 from various sources. Accordingly, other features of the system 10 operating under such “manual” and “signal” system control modes have to be generally similar or identical to each other. For example, the signal-controlled system 10 of this embodiment adopt control and actuating mechanisms which are similar or identical to those for the manually controlled system. Therefore, the actuator and control members of this signal control embodiment may effect the movements of the body member 20 and/or their configurational changes in a manner similar or identical to those members of the previous manual control embodiment.

It is also appreciated that the system 10 may have various configurations depending on which control mode such a system 10 may operate under. For example, when the system 10 is to operate strictly in the “manual control” mode but never in the “signal control” mode, such a system 10 may only include the above input units but may not have to operatively couple with the external A/V input and/or output units 91, 93. Conversely, when the system 10 is to operate strictly in the “signal control” mode but never in the “manual control” mode, such a system 10 may not include any input units but may be operatively coupled to such external A/V input and/or output units 91, 93. When desirable, the system 10 may be arranged to operate in both the “manual control” and “signal control” modes simultaneously or one at a time as well. In one example, the system 10 may include a selector which allows the user to select only one of the manual and signal control modes. In another example, the system 10 may be arranged to operate in the signal control mode by default and to receive the user inputs through such input units such that various signals carried or generated by the user inputs may be superposed onto the audiovisual (or control) signals, that such user inputs may interrupt or override the audiovisual (or control) signals, and the like. Further details of such control modes are to be provided in conjunction with FIGS. 2A and 2B.

In operation, the first and second units 21, 22 are fixedly or movably coupled to each other and form the body member 20. The system 10 is connected to an electric power outlet by a power supply cable (not included in the figure) or provided with a battery. The user may record various audiovisual (or control) signals and store such signals in the external A/V input unit 91. The user may instead use external storage media containing the audiovisual (or control) signals, where examples of such media may include, but not be limited to, magnetic tapes, magnetic disks, optical disks, microchips, and other data storage devices capable of temporarily or permanently storing analog or digital data therein, and retrieve such desired audiovisual (or control) signals through a suitable driver. The external A/V input unit 91 may also be an external communication device, an internet, and the like. Regardless of detailed configuration of such an external A/V input unit 91, the control member may obtain the desired signals therefrom and analyze various bases of the audiovisual signals, pulse trains of electric voltage and/or current, and the like. Depending upon the selected control mode of the system 10, the control member may receive the user input and superpose signals from the user input onto the audiovisual (or control) signals, modify or override such audiovisual (or control) signals based upon, in response to, and/or in synchronization with the user input, and the like. The user then inserts the head 21H of the first unit 21 into the internal cavity. Based upon, in response to or in synchronization with such audiovisual (or control) signals, the control member manipulates the actuator member to effect various movements of the first unit 21 of the body member 20, to change the configuration of such a member 20 and the like, without requiring the user to move her hands along or around the second unit 22 or without requiring the user to change the grip onto the second unit 22. For example, the control member may manipulate the actuator member to vary the curvature of the first unit 21, e.g., by increasing the acute angle (or decreasing the obtuse angle) defined between the straight and curved parts of the body member 20 and increasing a radius of circular movement of the first unit 21 as described in the bottom panel of FIG. 1A or vice versa. Alternatively, the control member may manipulate the actuator member to rotate in different speeds. Accordingly, the user may effect multiple movements of the body member 20 and also provide different stimuli based on, in response to or in synchronization with such audiovisual (or control) signals provided from various signal sources, without manually moving the body member 20 in the internal cavity for contacting the same part of the body member 20 with different portions of such a pelvic structure, and the like. Other configurational and/or operational characteristics of the system 10 of this embodiment are similar or identical to those of the system 10 of the previous embodiment. In addition, other configurational and/or operational characteristics of the system 10 of this embodiment of FIG. 1A are similar or identical to those of various systems which have been disclosed in the co-pending Applications.

In another exemplary embodiment of such an aspect of the present invention and as described in FIG. 1B, another exemplary pelvic relaxing system 10 is operatively coupled to at least one external A/V input unit 91 and/or at-least one external A/V output unit 93. The system 10 also includes a body member 20 with a first unit 21 and a second unit 22, an actuator member, and a control member with a first input unit 71F and a second input unit 71B, all of which are similar or identical to those of FIG. 1A. In contrary to that of FIG. 1A, however, the system 10 of FIG. 1B further includes one or more sensor units 75A, 75B which are incorporated into strategic locations of the first unit 21 of the body member 20. The sensor units 75A, 75B are preferably arranged to monitor various user inputs applied thereto through the pelvic opening and/or wall of the pelvic structure, i.e., the user may apply the user inputs by contacting or abutting such sensor units 75A, 75B with or against the pelvic opening and/or wall. For example, the first sensor unit 75A is disposed in the head 21H of the first unit 21 and, accordingly, may be used to monitor various dynamic patterns of the user input applied thereto through the G spot and/or deep pelvic walls positioned therearound. In contrary, the second sensor units 75B are rather disposed around the trunk 21T and, accordingly, may be used to monitor such dynamic patterns of the user input applied thereto through the clitoris and/or pelvic walls disposed closer to the opening of the pelvic structure. In general, such sensor units 75A, 75B may be arranged to monitor various dynamic patterns of the user inputs and to generate the sensing signals based upon, in response to, and/or in synchronization with such dynamic patterns. Accordingly, the sensor units 75A, 75B may be made of or include conventional sensors such as, e.g., force transducers, pressure sensors, velocity meters, accelerometers, and the like. Further configurational and/or operational characteristics of the sensor units 75A, 75B are typically identical or similar to those of the sensor units which have been disclosed in the co-pending Applications.

Contrary to those embodiments of FIG. 1A, the pelvic relaxing system 10 of FIG. 1B is arranged to obtain various sensing signals generated by such input and/or sensor units 71F, 71S, 75F, 75S and then to generate various sounds and/or images based upon, in response to or in synchronization with such signals. Therefore, the control member is arranged to receive the sensing signals from the input and/or sensor units 71F, 71S, 75F, 75S, to generate various audiovisual (or control) signals based on, in response to, and/or in synchronization with the sensing signals, and to deliver such audiovisual (or control) signals to the external A/V input unit 91 which may then convert such audiovisual (or control) signals into the desired sounds and/or images. The external A/V output unit 93 receives such sounds and/or images from the external A/V input unit 91 and plays the sounds and/or displaying the images. Alternatively, the control member may generate the sounds and/or images based upon, in response to or in synchronization with such sensing signals and deliver such sounds and/or images directly to the external A/V output unit 93. In the embodiment exemplified in FIG. 1B, the external A/V input unit 91 is a conventional computer, and the external A/V output unit 93 may be a conventional monitor therefor. When desirable, such an external A/V output unit 93 may also generate or obtain the audiovisual (or control) signals and then supply the signals to the internal A/V input unit of the system 10, where the external A/V input unit 91 may then be not required for various relaxing operations of the system 10 in such an embodiment.

The control member may be arranged to generate such audiovisual (or control) signals and/or such sounds and/or images in various modes. In one example, the control member may store multiple audiovisual (or control) signals in its storage unit and retrieve certain audiovisual (or control) signals based upon, in response to, and/or in synchronization with the sensing signals. Such audiovisual (or control) signals may then be sent to the external A/V input and/or output units 91, 93 which retrieve or generate the sounds and/or images corresponding to such audiovisual (or control) signals. In another example, the control member may store in its storage unit multiple content bases, voice bases, action bases, and/or background bases, and generates such audiovisual (or control) signals and/or sounds and images by combining multiple bases, by superposing one of bases onto the other, by synthesizing such audiovisual (or control) signals, sounds, and/or images from multiple bases of different persons, and the like. Therefore, such a system 10 may generate the sounds and/or images which may match various movements of a single part or multiple parts of its body member 20, which may match various movements of the entire body member 20, which may match various stimuli generated by the system 10, which may match a single portion or multiple portions of the pelvic structure receiving such stimuli, and the like. Other details of generating such signals, sounds, and/or images are also disclosed in the co-pending Applications.

In operation, the first and second units 21, 22 are fixedly or movably coupled with each other to form the body member 20, and the system 10 is connected to an electric power outlet with a power supply cable or provided with a battery. Similar to that of FIG. 1A, the user inserts the head 21H of the first unit 21 into the internal cavity, and pivots, swivels or otherwise manipulates the first input unit 71F which then generates the sensing signals and delivers the sensing signals to the control member. Based upon their dynamic patterns, the control member controls the actuator member to effect rotation of the first unit 21 about the longitudinal axis 25, thereby providing stimuli to such a circular portion of the pelvic structure. When the user desires different movements of the rotating first unit 21, she may apply different user inputs by pushing or swivelling the first input unit 71F in a preset direction without having to move her hands and/or without having to change the grip onto the second unit 22. The first input unit 71F generates different sensing signals in response to, based on or in synchronization with the user inputs and sends the signals to the control member which then controls the actuator member to change the curvature of the first unit 21 while increasing a radius of circular movement of the first unit 21 as described in the bottom panel of FIG. 1B or vice versa. Alternatively, the first input unit 71F may be mechanically coupled to the first unit 21 in order to bend the first unit 21 along one direction by bending the first input unit 71F in the same (or opposite) direction. When the user desires to change a speed and/or intensity of vibration of the first unit 21, she may change the dynamic pattern of the user inputs applied to the second input unit 71S. In response to, based on or in synchronization with such user inputs, the second input unit 71S generates different sensing signals and delivers the signals to the control member which may control the actuator member to rotate in different speeds. Accordingly, the user may effect multiple movements and provide different stimuli based upon, in response to or in synchronization with multiple user inputs. Not only through the input units 71F, 71S, the user may also apply various user inputs with the sensor units 75F, 75S. For example, the user may contact or abut such sensor units 75F, 75S with or against various portions of the pelvic structure which may then generate the sensing signals and delivers the signals to the control member. Based on their dynamic patterns, the control member may manipulate the actuator member to effect various movements of a single part or multiple parts of the first unit 21 and/or to change configuration of the body member 20, thereby providing various stimuli to the pelvic structure. When desirable, such a control member may intensify such stimuli of the same or different types as the user abuts the sensor units 75F, 75S with greater force and/or for a longer period of time.

Depending upon, in response to or in synchronization with such user inputs received by such input units 71F, 71S and/or sensor units 75F, 75S, the control member may generate such audiovisual (or control) signals or may generate such sounds and/or images. In one example, the control member may store multiple control signals in its storage unit and retrieve certain control signals based upon, in response to, and/or in synchronization with the user inputs. Such control signals may be sent to the external A/V input and/or output units 91, 93 which generate the sounds and/or images corresponding to such control signals. When the input and/or sensor units 71F, 71S, 75F, 75S receive different user inputs with different dynamic patterns, the control member may generate different control signals and control the external A/V units 91, 93 to generate different sounds and/or images which correspond to different user inputs. In another example, the control member may store multiple audiovisual signals in its storage unit and retrieve certain audiovisual signals. The external A/V output unit 93 receives the retrieved audiovisual signals and then generates the sounds and images carried by such audiovisual signals. When the input and/or sensor units 71F, 71S, 75F, 75S receive different user inputs defining different dynamic patterns, the control member may generate different audiovisual signals and control the external A/V units 91, 93 to generate different sounds and images corresponding to different user inputs. In another example, the control member may store multiple content bases, voice bases, action bases, and/or background bases in its storage unit, and generates certain audiovisual signals through combining multiple bases of different audiovisual signals, superposing at least one of such bases onto the others, replacing at least one of such bases of a certain audiovisual signal by at least one another of such bases of another signal, synthesizing multiple bases into a desired audiovisual signal, and the like. The external A/V output unit 93 receives the combined, superposed, and/or synthesized signals from the control member and generates such sounds and images carried by such signals. When the input and/or sensor units 71F, 71S, 75F, 75S receive different user inputs defining different dynamic patterns, the control member may combine, superpose, and/or synthesize different control signals and control the external A/V units 91, 93 to generate different sounds and/or images which correspond to different user inputs. Accordingly, such a system 10 may generate the sounds and/or images which may match various movements of a single or multiple parts of its body member 20, which may match various movements of the entire body member 20, which may match various stimuli generated by the system 10, which may match a single portion or multiple portions of the pelvic structure receiving such stimuli, and so on. Other configurational and/or operational characteristics of the system 10 of FIG. 1B may be similar or identical to those of the systems of both of the embodiments of FIG. 1A and various systems which have been disclosed in the co-pending Applications.

In another exemplary embodiment of such an aspect of the present invention and as described in FIG. 1C, an exemplary pelvic relaxing system 10 operatively couples with at least one external A/V input and/or output units (not shown in the figure). The system 10 includes a body member 20 with a first unit 21 and a second unit 22, an actuator member incorporated inside such units 21, 22 (thus not included in this figure), and a control member including an input unit 71 and other units included inside the body member 20 (thus not shown in this figure). Similar to those of FIGS. 1A and 1B, the first unit 21 is generally elongated, forms a part insertable into the internal cavity during use, and also includes the head 21H, trunk 21T, and base 21B, while the second unit 22 is distally coupled to the first unit 21 and provides a grip to an user during use. The first and second units 21, 22 may movably, fixedly or releasably couple with each other and similar to those of FIGS. 1A and 1B.

The first unit 21 also defines an annular bulge 23 between its trunk 21T and base 21B. Such a bulge 23 generally protrudes outwardly and is also shaped and/or sized so that the bulge 23 abuts the inner wall of the internal cavity of the pelvic structure when inserted through the opening of the pelvic structure. Therefore, the bulge 23 may retain itself and other parts of the first unit 21 proximal thereto inside the internal cavity once inserted thereinto and also facilitate hand-free operation of the system 10. When desirable, the bulge 23 may be arranged to define variable shapes and/or sizes to allow the user to select a desirable shape and/or size depending upon detailed anatomy of her pelvic structure.

In contrary to the first units of FIGS. 1A and 1B, the middle part of the first unit 21 of FIG. 1C is arranged to translate along the longitudinal axis of the body member 20 with respect to the rest of the first unit 21, while maintaining the lengths of the heat 21H and base 21B thereof. Accordingly, the first unit 21 may change its length as its trunk 21T translates between its off-state (i.e., when the first unit 21 defines a minimum length) and its on-state (i.e., when the first unit 21 translates or deforms into a maximum length). In the embodiment of FIG. 1C, the translating trunk 21T is shaped as a bellow so that distances between peaks (or ridges) approach a minimum when the first unit 21 is in its off-state and a maximum when such a first unit 21 is in its on-state, thereby varying the length of the entire first unit 21. Accordingly, such a system 10 may vary the length of the inserted part of the first unit 21 during its use while retaining the inserted and translating part of the first unit 21 inside the internal cavity by abutting the internal walls of the pelvic structure with the above bulge 23 of the first unit 21. Although not shown in FIG. 1C, the actuator member is preferably arranged to generate translating movements of the first unit 21 by conventional various mechanisms of converting rotational movement of its driver unit into the translating movements, where examples of such mechanisms are provided in detail in the above prior art patents and/or publications incorporated herein in their entireties by reference. Such a translating trunk 21T may be formed as and/or covered by the bellows as depicted in FIG. 1C or, in the alternative, may be made of an elastic cover which may vary its length between the off- and on-state of the first unit 21.

The second unit 22 defines therealong at least one track 72T which extends linearly along the longitudinal axis of the body member 20. The input unit 71 is movably disposed inside the track 72T in order to move along the track 72T while being guided thereby. The input unit 71 also defines a circular receiving area and receives through such an area the user input which moves the input unit 72T along the track 72T. In the embodiment of FIG. 1C, the input unit 71 is arranged to manipulate a displacement or a length of travel of the trunk 21T of the first unit 21 between its off-and on-state, e.g., the length of travel is kept to the minimum when the input unit 71 is positioned in a distal end of the track 72T and to the maximum when the input unit 71 is moved to a proximal end of the track 72T. The input unit 71 may further define at least one more state between the proximal and distal ends of the track 72T such that the length of travel may take a value between such a maximum and minimum. Multiple states may also be defined along the track 72T intermittently or continuously so that the user may manipulate the length of travel of the trunk between the above maximum and minimum values intermittently or continuously. In any of such examples, the input unit 71 generates various sensing signals, and the control member receives the sensing signals and manipulates actuating and conversion mechanisms of the driver unit of the actuator member, thereby adjusting the lengths of travel accordingly.

The input unit 71 may be equipped with an auxiliary mechanism for receiving a different type of user inputs as well. For example, the input unit 71 may be arranged to move vertically while defining at least two states which are generally independent of those states defined along the track 72T. For each of the states defined vertically with respect to the track 72T, the input unit 71 may generate the sensing signals based upon which the control member may manipulate the actuator member to effect different movements, the same movement with different dynamic features, and so on. Therefore, the control member may adjust the length of travel of the first unit 21 based on the disposition of the input unit 71 along the track 72T, while controlling a speed of translation, intensity of vibration, and the like, with the vertical disposition of the input unit 71. In another example, the input unit 71 may be arranged to operate as a conventional joystick which may pivot or swivel around each state along the track 72T while defining at least two different states based on disposition of the input unit 71 with respect to the track 72T. In another example, the input unit 71 may incorporate the variable-resistance mechanism of the input units of FIG. 1A so that such an unit 71 generates another set of sensing signals based on an amplitude of the user input applied thereto, a direction thereof, a duration thereof, and the like, and that the control member receives such signals and manipulate the actuator member to effect different movements, the same movement with different dynamic features, and so on. In another example, the variable-resistance input unit of FIGS. 1A and 1B may be incorporated in the second unit 22 in addition to the input unit 71 of FIG. 1C. Any other input units of the co-pending Applications may be included in the second unit 22 as well for controlling various features of various movements and/or for changing the configuration of the first unit 21.

When desirable, the system 10 may also include various sensor units as exemplified in FIG. 1B and generate the sensing signals with the input and/or sensor units for playing various sounds and/or displaying various images based on, in response to or in synchronization with the signals. Therefore, the control member is arranged to receive the sensing signals from such input and/or sensor units, to generate various audiovisual (or control) signals based on, in response to or in synchronization with the sensing signals, and to deliver such audiovisual (or control) signals to the external A/V input unit which may then convert such audiovisual (or control) signals into the desired sounds and/or images. The external A/V output unit receives such sounds and/or images from the external A/V input unit and plays the sounds and/or displaying the images. In the alternative, the control member may generate the sounds and/or images based upon, in response to or in synchronization with such sensing signals and deliver such sounds and/or images directly to the external A/V output unit. When desirable, such an external A/V output unit may also generate or obtain the audiovisual (or control) signals and supply the signals to the internal A/V input unit of the system 10, where the external A/V input unit may then be not required for various relaxing operations of the system 10 in such an embodiment. The control member may be arranged to generate such audiovisual (or control) signals and/or such sounds and/or images in various modes as described in conjunction with FIG. 1B.

In operation, the first and second units 21, 22 are fixedly or movably coupled with each other to form the body member 20, and the system 10 is connected to an electric power outlet with a power supply cable (not shown in the figure) or provided with a battery. The user then inserts the head 21H of the first unit 21 into the internal cavity. When the user positions the input unit 71 along one position along the track 72T as exemplified in the top and middle panels of FIG. 1C and moves the input unit 71 to a preset state, the input unit 71 generates the sensing signals based on position thereof along the track 72T and delivers such signals to the control member. Based upon dynamic patterns of the user input, the control member may manipulate the actuator member to effect translation of the first unit 21 of a preset length of travel at a preset speed as determined by the user input, thereby providing stimuli to the pelvic structure. When the user desires a different length of travel of the first unit 21, she may move the first input unit 71 to another position along the track 72T as exemplified in the bottom panel of FIG. 1C. The control member receives different sensing signals and manipulates the actuator member to adjust the conversion mechanisms of its driver unit and to effect a different length of travel. In the examples of FIG. 1C, the input unit 71 is moved from its first state for the minimum length of travel (the top panel) to its second state for the maximum length of travel (the bottom panel), thereby allowing the head 21H and trunk 21T of the first unit 21 to penetrate into a greater depth of such a pelvic structure. When the input unit 71 is equipped with the auxiliary mechanism for receiving different user inputs, the user may apply different user inputs by simply pushing or swivelling the input unit 71F along a preset direction without having to move her hands along or around the second unit 22 and/or without having to change the grip onto the second unit 22. The input unit 71 then generates a different set of sensing signals in response to, based upon or in synchronization with the user inputs and sends the signals to the control member which manipulates the actuator member to effect different movements, to change dynamic features of the same movement, to change the configuration of the first unit 21, and the like. Accordingly, the user may effect multiple movements of the first unit 21 and provide different stimuli in response to, based upon or in synchronization with multiple user inputs. Other configurational and/or operational characteristics of such “manual system control mode” are similar or identical to those of the same control mode of FIG. 1A and those of various systems which have been disclosed in the co-pending Applications.

In the alternative, the system 10 of FIG. 1C may operate in the “signal system control mode” as well. The first and second units 21, 22 are similarly coupled to each other, while one or more sensor units of FIG. 1B are incorporated into the first unit 21. The head 21H of the first unit 21 is inserted into the internal cavity of the structure, and the actuator member effects the translational movement of the first unit 21 of a preset length of travel at a preset speed determined by the position of the input unit 71 along the track 72T. The sensor units generate various sensing signals based on, in response to, and/or in synchronization with various user inputs applied thereto through the structure. The control member receives such sensing signals from the input and/or sensor units, and generates audiovisual (or control) signals or, alternatively, generates sounds and/or images. In one example, such a control member may store multiple control signals in its storage unit and retrieve certain control signals based on, in response to, and/or in synchronization with the user inputs. Such control signals may be sent to the external A/V input and/or output units which generate the sounds and/or images corresponding to the control signals. When the input and/or sensor units receive different user inputs with different dynamic patterns, the control member may generate different control signals and control the external A/V units to generate different sounds and/or images which correspond to different user inputs. In another example, the control member may also store multiple audiovisual signals in its storage unit and retrieve certain audiovisual signals. The external A/V output unit receives such retrieved audiovisual signals and generates the sounds and/or images carried by such audiovisual signals. When the input and/or sensor units receive different user inputs with different dynamic patterns, the control member may generate different audiovisual signals and manipulate the external A/V input and/or output units to generate different sounds and/or images corresponding to different user inputs. In another example, the control member may store multiple content bases, voice bases, action bases, and/or background bases in its storage unit, and generates certain audiovisual signals through combining multiple bases of different audiovisual signals, superposing at least one of such bases onto the others, replacing at least one of the bases of a certain audiovisual signal by at least one another of the bases of another signal, synthesizing multiple bases into a desired audiovisual signal, and so on. Such an external A/V output unit receives the combined, superposed, and/or synthesized signals from the control member and generates such sounds and images carried by such signals. When the input and/or sensor units receive different user inputs defining different dynamic patterns, such a control member may combine, superpose, and/or synthesize different control signals and control the external A/V units to generate different sounds and/or images which correspond to different user inputs. Therefore, the system 10 may generate the sounds and/or images which may match various movements of a single or multiple parts of its body member 20, which may also match various movements of the entire body member 20, which may instead match various stimuli provided by the system 10, which may match a single portion or multiple portions of the pelvic structure receiving such stimuli, and the like. Further configurational and/or operational characteristics of the system 10 of FIG. 1C in the “signal system control mode” may be similar or identical to those of the systems of FIG. 1B as well as those of various systems which have been disclosed in the co-pending Applications.

In another exemplary embodiment of such an aspect of the present invention and as described in FIG. 1D, an exemplary pelvic relaxing system 10 operatively couples with at least one external A/V input and/or output units (not shown in the figure). The system 10 includes a body member 20 with a first unit 21 and a second unit 22, an actuator member incorporated inside such units 21, 22 (thus not included in this figure), and a control member including an input unit 71 and other units included inside the body member 20 (thus not shown in this figure). Similar to those of FIGS. 1A and 1B, the first unit 21 is generally elongated, forms a part insertable into the internal cavity during use, and also includes the head 21H, trunk 21T, and base 21B, while the second unit 22 is distally coupled to the first unit 21 and provides a grip to an user during use. The first and second units 21, 22 may movably, fixedly or releasably couple with each other and similar to those of FIGS. 1A and 1B.

The first unit 21 also includes multiple electrodes 24 which are preferably exposed through its surface and also arranged to deliver an electric voltage or current therethrough. More particularly, at least two of such electrodes 24 are electrically connected so that the electric voltage or current may flow from at least one of the electrodes 24 to at least another thereof along one or both directions. To this end, such electrodes 24 are wired in a preset arrangement or, in the alternative, wiring among the electrodes 24 may be adjusted by the control member. In general, an amplitude of the electric voltage or current is arranged to be on the order of a minimum value for evoking a sensation or contraction of the pelvic muscles, to be slightly less than such a minimum value or to exceed the minimum value by a preset amplification factor, where examples of the amplitudes are well known in the art and provided in general anatomy or physiology textbooks. The control member may generate the electric voltage or current having the same amplitude or may alternatively vary the amplitudes of such voltage or current based on, in response to or in synchronization with various dynamic patterns of the user input. Such electrodes 24 may be distributed over the surface of the first unit 21 in a preset uniform arrangement. In the alternative, the electrodes 24 may also be distributed not uniformly but concentrated in strategic locations of the external surface of the first unit 21. In yet another alternative and regardless of such arrangements the electrodes 24, the control member may also be arranged to recruit a limited number of but not all of such electrodes 24 to provide preset stimuli to a single or multiple portions of the pelvic structure. In the embodiment of FIG. 1D, multiple electrodes 24 distributed in an uniform arrangement over the entire surface of such a first unit 21. It is appreciated that the system 10 may include various electrodes 24 with various configurations. In one example and as shown in the figure, the electrodes 24 may have circular conducting areas. In another example, such electrodes may define noncircular contacting areas such as, e.g., triangular, rectangular or other polygonal areas. In another example, the electrodes may define elongated shapes as well. Such electrodes 24 may extend along various directions as well. For example, the electrodes may extend parallel to the longitudinal axis of the first unit 21, perpendicular thereto, at a preset angle thereto, and the like. When desirable, the electrodes may wind around a circumference of the first unit 21 while defining shapes of concentric rings, wind the circumference helically, and the like.

The second unit 22 includes a first input unit 71F which is exposed around the second unit 22 for providing direct access to the user and receives the user input through at least a substantial area thereof. The first input unit 71F is incorporated into a center part of the second unit 22, while forming vacant areas between itself 71F and proximal and distal ends of the second unit 22. Therefore, such a first input unit 71F shares an area of the second unit 22 which is also used to define the grip for the user. Similar to the second input units of FIGS. 1A and 1B, the first input unit 71F is similarly arranged to sense various dynamic patterns of mechanical or electrical contact with the user, to sense those of force applied thereto by the user, and the like, and to generate sensing signals based on, in response to or in synchronization with such dynamic patterns of the user inputs. Further configurational and/or operational characteristics of the first input unit 71 are similar or identical to those of the second input units of FIGS. 1A and 1B and to those of other similar input units of the co-pending Applications.

The second unit 22 include multiple second input units 71S which are also exposed around the second unit 22 for providing direct access to the user and receive the user inputs therethrough. Such second input units 71S are generally incorporated inside the first input unit 71F and share the area of the second unit 22 which is also used to define the grip for the user. Therefore, the user may control both of the first and second input units 71F, 71S without having to change the grip or move the hand. Such second input units 71S are also arranged to monitor various dynamic patterns of mechanical or electrical contact with the user and/or to monitor those of force applied thereto, and then to generate sensing signals based upon, in response to or in synchronization with such dynamic patterns of the user inputs. Such second input units 71S are preferably disposed in an arrangement corresponding to an arrangement of the electrodes 24 of the first unit 21 so that application of the user inputs to one of the second input units 71S causes a certain electrode 24 or a set of multiple preset electrodes 24 to deliver the electric voltage or current to the pelvic structure. In one example, each second input unit 71S may be arranged to cause only one electrode 24 to deliver the voltage or current to a surrounding tissue of the pelvic structure or to cause a pair of or more than two electrodes 24 to flow the electric current therebetween. In this example, such a second unit 22 preferably includes an equal number or a similar number of the second input units 71S as the electrodes 24. In another example, each second input unit 71S may cause a group of electrodes 24 to deliver the electric voltage or current to such a pelvic structure or to deliver such therebetween. Other examples may also be embodied as far as the second input units 71S may receive the user inputs defining a certain spatial pattern and then cause the electrodes 24 to deliver the electric voltage or current in the same or at least similar spatial pattern. The control member may synchronize the first input unit 71F with the second input unit 71S as well so that the amplitudes of the electric voltage or current delivered by the electrodes 24 may be controlled by various dynamic patterns of the user input applied to the first input unit 71F. Thus, such a control member may manipulate whether the electric voltage or current may be delivered from one electrode 24 primarily to the structure or to adjacent electrodes 24 depending on the user input received by the first input unit 71F, may manipulate the amplitudes of the electric voltage or current based on the user inputs received by the first input unit 71F, and the like.

The input units 71F, 71S may be equipped with an auxiliary mechanism for receiving a different type of user inputs as well. For example, either of the input units 71F, 71S may be arranged to move vertically while defining at least two states which are generally independent of those states defined hereinabove. Such input units 71F, 71S may generate the sensing signals based on which the control member may manipulate the actuator member to effect different movements, the same movement with different dynamic features, and the like. Accordingly, the actuator member may be arranged to effect vibration, translation or other movements of the first unit, while controlling various dynamic features of the movements through the auxiliary mechanism. When desirable, the system 10 may include various sensor units as exemplified in FIG. 1B, and generate the sensing signals with the input and/or sensor units for playing various sounds and/or displaying various images based on, in response to, and/or in synchronization with the signals. Accordingly, the control member is arranged to receive the sensing signals from such input and/or sensor units, to generate various audiovisual (or control) signals based on, in response to, and/or in synchronization with the sensing signals, and to deliver such audiovisual (or control) signals to the external A/V input unit which may then convert such audiovisual (or control) signals into the desired sounds and/or images. The external A/V output unit may receive such sounds and/or images from the external A/V input unit and play the sounds and/or display the images. In the alternative, the control member may generate the sounds and/or images based upon, in response to, and/or in synchronization with such sensing signals and deliver such sounds and/or images directly to the external A/V output unit. When desirable, such an external A/V output unit may also generate or obtain the audiovisual (or control) signals and supply the signals to the internal A/V input unit of the system 10, where the external A/V input unit may then be not required for various relaxing operations of the system 10 in such an embodiment. The control member may also be arranged to generate such audiovisual (or control) signals and/or such sounds and/or images in various modes as described in conjunction with FIG. 1B.

In operation, the first and second units 21, 22 are fixedly or movably coupled with each other to form the body member 20, and the system 10 is connected to an electric power outlet with a power supply cable (not shown in the figure) or provided with a battery. The user then inserts the head 21H of the first unit 21 into the internal cavity. As the user holds the second unit 22, she also contacts or presses one or more of the second input units 71S which then generate the sensing signals based on the spatial pattern of the user input. As described above, such second input units 71S are preferably arranged to generate such signals which also carry or at least reflect such spatial pattern of the user input. Based upon dynamic (i.e., spatial as well as temporal) patterns of such user inputs, the control member may deliver the electric voltage or current through a preset number of electrodes 24 based on the spatial pattern of the user input while causing sensation or contraction of muscles of the structure contacting with such electrodes 24, thereby providing electric stimuli to the pelvic structure. When the user desires different electric stimuli, she may contact and/or press a different set of the second input units 71S without having to change the grip of the second unit 22 or without having to move the hand. The control member then receives different sensing signals and manipulates different electrodes 24 to deliver the voltage or current to the pelvic structure, thereby effecting different electric stimuli. When the second input units 71S are equipped with such an auxiliary mechanism for receiving different user inputs, the user may apply different user inputs by simply pushing or swivelling the second input units 71S without having to move her hand along or around the second unit 22 or without having to change the grip onto the second unit 22. The second input units 71S then generate a different set of sensing signals in response to, based upon or in synchronization with the user inputs and sends the signals to the control member which delivers the electric voltage or current to different electrodes 24, to change spatial or temporal features of such electric excitation, to change the amplitudes, duration or direction of such voltage or current, and the like. Accordingly, the user may effect multiple movements of such a first unit 21 and provide different stimuli in response to, based on or in synchronization with multiple user inputs. The user may also apply various user inputs to the first input unit 71F which operates in a manner similar or identical to those of FIGS. 1A and 1B. Further configurational and/or operational characteristics of such “manual system control mode” are also similar or identical to those of the same control mode of FIGS. 1A and 1C and those of various systems which have been disclosed in the co-pending Applications.

In the alternative, the system 10 of FIG. 1D may operate in the “signal system control mode” as well. The first and second units 21, 22 are similarly coupled to each other, while one or more sensor units of FIG. 1B are incorporated into the first unit 21. The head 21H of the first unit 21 is inserted into the internal cavity of the structure, and the control member provides electric stimuli with the electrodes 24 of the first unit 21. The sensor units generate various sensing signals based upon, in response to, and/or in synchronization with various user inputs applied thereto through the structure. The control member receives such sensing signals from the input and/or sensor units, and generates audiovisual (or control) signals or, alternatively, generates sounds and/or images as explained in conjunction with such control modes of FIGS. 1B and 1C. Accordingly, the system 10 may generate the sounds and/or images matching various movements of a single part or multiple parts of its body member 20, matching various movements of the entire body member 20, matching various stimuli provided by the system 10, matching a single portion or multiple portions of the pelvic structure receiving such stimuli, and the like. Other configurational and/or operational characteristics of the system 10 of FIG. 1D under the “signal system control mode” are similar or identical to those of the systems of FIGS. 1B and 1C and those of various systems which have been disclosed in the co-pending Applications.

In another exemplary embodiment of this aspect of the invention and as depicted in FIG. 1E, an exemplary pelvic relaxing system 10 includes a body member 20 with a first unit 21 and a second unit 22, an actuator member incorporated inside the first and second units 21, 22 of the body member 20, and a control member including a first input unit 71F and a second input unit 71B, each of which may be similar or identical to a corresponding member or unit of FIGS. 1A and 1B. Although not included in the figure, the system 10 may be coupled to at least one external A/V input and/or output units 91, 93 which have been described in conjunction with FIGS. 1A and 1B. In contrary to those of FIGS. 1A and 1B, however, the first unit 21 also includes a bulge 23 which is formed in the base 21B and which is strategically shaped and/or sized to contact a clitoris of the pelvic structure. An entire part or at least a part of the bulge 23 is thereby arranged to effect vibration and/or other movements so as to deliver stimuli to the clitoris. When desirable, the bulge 23 may be arranged to translate, rotate, pivot, swivel, vibrate or otherwise move between multiple states while changing its disposition, orientation, and the like. Accordingly, such a bulge 23 may provide the stimuli to different portions of the pelvic structure, may provide different stimuli to the same or different portions of the structure, and the like. The bulge 23 may also be arranged to deform when abutted by the structure, when manipulated by the control member, and the like.

The first input unit 71F is movably incorporated onto a distal end of the second unit 22 and also oriented so that the user may apply the user input thereto through at least an area of the first input unit 71F vertically, horizontally, at an angle or angularly. The first input unit 71F moves or operates similar to conventional joysticks and is generally identical to those of FIGS. 1A and 1B. The second input unit 71S is exposed through the second unit 22 and operates typically similar to those of FIGS. 1A and 1B. Contrary to those of FIGS. 1A and 1B, the control member is arranged to manipulate disposition and/or orientation of the bulge 23 with respect to the rest of the first unit 21 so that manipulating such a first input unit 71F in one direction manipulates the bulge 23 to move or deform along the same or opposite direction. In the embodiment of FIG. 1E, the control member is arranged to adjust a height of the bulge 23 when the first input unit 71F is pivoted about the longitudinal axis of the body member 20. The first input unit 71F may be arranged to manipulate the bulge 23 in various embodiment. In one example, the first input unit 71F is mechanically coupled to the bulge 23 so that movement of the first input unit 71F from its off-state may move or swivel the bulge 23 accordingly. In another example, the first input unit 71F generates the sensing signals, and the control member then receives the signals and activate the actuator member to dispose and/or orient the bulge 23 in response to such signals.

In operation, the first and second units 21, 22 are fixedly or movably coupled with each other to form the body member 20, and the system 10 is connected to an electric power outlet with a power supply cable (not included in the figure) or provided with a battery. The user inserts the head 21H of the first unit 21 into the internal cavity. In response to the user input, the second input unit 71S begins to generate the sensing signals, and the control member effects various movements of a single part or multiple parts of the body member 20 to deliver such stimuli to a single or multiple portions of the pelvic structure. When the user desires different stimuli, she may press a different area of the second input unit 71S and/or apply the user inputs with different dynamic patterns without changing the grip of the second unit 22 or without moving the hand. The control member may then receive different sensing signals and manipulate the actuator member for effecting different movements of the body member 20 and/or for changing the configuration thereof. During such use, the user may provide the user inputs to the first input unit 71F. In response to such user inputs, the control member may adjust disposition and/or orientation of the bulge 23, thereby delivering various movements and providing various stimuli to the clitoris. When the second input unit 71S is equipped with the auxiliary mechanism for receiving different user inputs, the user may apply different user inputs through simply pushing or swivelling the second input unit 71S without having to move her hand along or around the second unit 22 or without having to change the grip onto the second unit 22. The second input unit 71S generate a different set of sensing signals in response to, based upon or in synchronization with such user inputs and sends the signals to the control member which effects different movements or effects the same movement with different dynamic features. Accordingly, the user may effect multiple movements of such a first unit 21 and provide different stimuli in response to, based on or in synchronization with multiple user inputs. Other configurational and/or operational characteristics of such “manual system control mode” are also similar or identical to those of the same control mode of FIGS. 1A to 1D and those of various systems which have been disclosed in the co-pending Applications.

In the alternative, the system 10 of FIG. 1E may operate in the “signal system control mode” as well. The first and second units 21, 22 are similarly coupled to each other, while one or more sensor units of FIG. 1B are incorporated into the first unit 21. The head 21H of the first unit 21 is inserted into the internal cavity of the structure, and the control member provides electric stimuli with the electrodes 24 of the first unit 21. The sensor units generate various sensing signals based upon, in response to, and/or in synchronization with various user inputs applied thereto through the structure. The control member receives such sensing signals from the input and/or sensor units, and generates audiovisual (or control) signals or, alternatively, generates sounds and/or images as explained in conjunction with such control modes of FIGS. 1B to 1D. Accordingly, the system 10 may generate the sounds and/or images matching various movements of a single part or multiple parts of its body member 20, matching various movements of the entire body member 20, matching various stimuli provided by the system 10, matching a single portion or multiple portions of the pelvic structure receiving such stimuli, and the like. Other configurational and/or operational characteristics of the system 10 of FIG. 1E under the “signal system control mode” are similar or identical to those of such systems of FIGS. 1B to 1D and those of various systems which have been disclosed in the co-pending Applications.

In another exemplary embodiment of this aspect of the invention and as depicted in FIG. 1F, an exemplary pelvic relaxing system 10 includes a body member 20 with a first unit 21 and a second unit 22, an actuator member incorporated inside the first and second units 21, 22 of the body member 20, and a control member including a first input unit 71F and a second input unit 71B, each of which may be similar or identical to a corresponding member or unit of FIGS. 1A and 1B. Although not included in the figure, the system 10 may be coupled to at least one external A/V input and/or output units 91, 93 which have been described in conjunction with FIGS. 1A and 1B. In contrary to those of FIGS. 1A and 1B, however, the first unit 21 also includes multiple bulges 23 which are included in the base 21B and strategically shaped and/or sized to contact a G-spot of the pelvic structure. Such bulges 23 may be arranged to effect vibration, translation, rotation, and/or other movements so as to deliver stimuli to the G-spot. When desirable, the bulges 23 may also be arranged to translate, rotate, pivot, swivel, vibrate or otherwise move between multiple states while changing their disposition, orientation, arrangement, and the like. Accordingly, such bulges 23 may provide the stimuli to different portions of the structure, may provide different stimuli to the same or different portions of the structure, and the like. The bulges 23 may also be arranged to deform when abutted by the structure, when manipulated by the control member, and the like.

The first input unit 71F is incorporated onto the second unit 22 and oriented such that the user may apply the user input through at least an area of the first input unit 71F vertically, horizontally, at an angle or angularly. The first input unit 71F is preferably arranged to sense a location thereof to which the user applies the user input, e.g., similar to conventional touch pads or touch screens, and then to generate sensing signals which carry information regarding spatial features of such user inputs. The second input unit 71S is exposed through the second unit 22 and operates typically similar to those of FIGS. 1A and 1B. Contrary to those of FIGS. 1A and 1B, the control member is arranged to manipulate disposition and/or orientation of the bulges 23 with respect to the rest of the first unit 21. Accordingly, applying the user input onto the first input unit 71F in one direction manipulates the bulges 23 to move or deform along the same (or opposite) direction. In the embodiment of FIG. 1F, the control member is arranged to adjust disposition of the bulges 23 when the user input is applied to different areas of the first input unit 71F. Such a first input unit 71F may be arranged to manipulate the bulges 23 in various embodiment. In one example, the first input unit 71F is mechanically coupled to the bulges 23 such that movement of the first input unit 71F from its off-state may move the bulges 23 accordingly. In another example, the first input unit 71F generates the sensing signals, and the control member then receives the signals and activate the actuator member to dispose and/or orient the bulges 23 as a response to such signals. Similar to other embodiments of the above figures, the control member may be provided with an auxiliary mechanism for receiving a different type of user inputs as well. For example and as exemplified in the bottom panel of FIG. 1F, the first input unit 71F may be arranged to be depressed in response to the user input and to generate a different set of sensing signals which may manipulate the actuator member to effect another movement. When desirable, the auxiliary mechanism may also be spatially sensitive such that the first input unit 71F may generate different sets of sensing signals in response to depression of different areas thereof.

In operation, the first and second units 21, 22 are fixedly or movably coupled with each other to form the body member 20, and the system 10 is connected to an electric power outlet with a power supply cable (not included in the figure) or provided with a battery. The user inserts the head 21H of the first unit 21 into the internal cavity. In response to the user input, the second input unit 71S begins to generate the sensing signals, and the control member effects various movements of a single part or multiple parts of the body member 20 to deliver such stimuli to a single or multiple portions of the pelvic structure. When the user desires different stimuli, she may press a different area of the second input unit 71S and/or apply the user inputs with different dynamic patterns without changing the grip of the second unit 22 or without moving the hand. The control member may then receive different sensing signals and manipulate the actuator member for effecting different movements of the body member 20 and/or for changing the configuration thereof. During such use, the user may provide the user inputs to the first input unit 71F. In response to such user inputs, the control member may adjust disposition and/or orientation of one or all of such bulges 23, thereby delivering various movements and providing various stimuli to the G-spot. When the first input unit 71F includes the auxiliary mechanism to receive different user inputs, the user may apply different user inputs through simply pushing or swivelling the first input unit 71F without moving her hand along the second unit 22 or without changing the grip onto the second unit 22. The first input unit 71F generate a different set of sensing signals in response to, based upon or in synchronization with such user inputs and sends the signals to the control member which effects different movements or the same movement with different dynamic features. Thus, the user may effect multiple movements of such a first unit 21 and provide different stimuli in response to, based upon or in synchronization with multiple user inputs. Other configurational and/or operational characteristics of such “manual system control mode” are also similar or identical to those of the same control mode of FIGS. 1A to 1E as well as those of various systems which have been disclosed in the co-pending Applications.

In the alternative, the system 10 of FIG. 1F may operate in the “signal system control mode” as well. The first and second units 21, 22 are similarly coupled to each other, while one or more sensor units of FIG. 1B are incorporated into the first unit 21. The head 21H of the first unit 21 is inserted into the internal cavity of the structure, and the control member provides electric stimuli with the electrodes 24 of the first unit 21. The sensor units generate various sensing signals based upon, in response to, and/or in synchronization with various user inputs applied thereto through the structure. The control member receives such sensing signals from the input and/or sensor units, and generates audiovisual (or control) signals or, alternatively, generates sounds and/or images as explained in conjunction with such control modes of FIGS. 1B to 1E. Accordingly, the system 10 may generate the sounds and/or images matching various movements of a single part or multiple parts of its body member 20, matching various movements of the entire body member 20, matching various stimuli provided by the system 10, matching a single portion or multiple portions of the pelvic structure receiving such stimuli, and the like. Other configurational and/or operational characteristics of the system 10 of FIG. 1F under the “signal system control mode” are similar or identical to those of such systems of FIGS. 1B to 1E and those of various systems which have been disclosed in the co-pending Applications.

Configurational and/or operational variations and/or modifications of the above embodiments of the exemplary systems and various members thereof described in FIGS. 1A through 1F also fall within the scope of this invention.

The body member and its units may be fabricated similar or identical to shapes and/or sizes of any conventional pelvic relaxing devices. Accordingly, the first unit may have various shapes, sizes, and curvatures which may be constant along an entire portion of a longitudinal axis of the first unit or may vary therealong. Further details of such shapes, sizes, curvature, and other configurations are provided in the above prior art patents and/or publications incorporated herein by reference. Similarly, the second unit may have various shapes, sizes, and curvatures which may also be constant along the entire portion of the axis or may also vary therealong. It is appreciated, however, that handles of the conventional pelvic relaxing devices are generally longer in order to accommodate a space for the grip and another space for incorporating various switches. In contrary, the pelvic relaxing system of this invention incorporates the input unit disposed in the same area as the grip therefor and, therefore, the second unit of such a system may typically be shorter than its conventional counterparts. Such a body member and its first and second units may also be made of and/or include materials as employed in any conventional pelvic relaxing devices. Accordingly, the first unit may be made of and/or include flexible or rigid materials, soft or hard materials, composite materials, and the like. When desirable, the first unit may be arranged to exhibit different mechanical properties in different parts thereof. The first unit may further form at least one internal lumen in which various members and/or units of the system may be disposed. The second unit may also be made of and/or include flexible or rigid materials, soft or hard materials, as long as such an unit may form the grip for the user.

Although the pelvic relaxing systems of FIGS. 1A to 1F exemplify combinations of various first units, second units, input units, and sensor units with various external A/V input and A/V output units, these units may be incorporated in different combinations as well. For example, the external A/V input and output units of the system of FIG. 1A or 1B may be used in conjunction with any of such systems of FIGS. 1C to 1F, and their body members 20 may include any of the input and/or sensor units in the same or different locations along the first and/or second units. The system may incorporate different combinations of various body, actuator, and/or control members as will be described in greater detail below. Other details of various members and/or units of such pelvic relaxing systems of FIGS. 1A to 1F are provided in the co-pending Applications.

In another aspect of the present invention, an exemplary pelvic relaxing system may include at least one body member, at least one actuator member, at least one control member, and at least one power (supply) member. FIGS. 2A and 2B depict schematic diagrams of various functional members and units of an exemplary pelvic relaxing system according to the present invention. A typical pelvic relaxing system 10 is comprised of at least one body member 20, at least one actuator member 60, at least one control member 70, and at least one power (supply) member (not included in these figures) which may be a wire and plug assembly for receiving AC power from an electric outlet or may instead be a dry-cell battery or a rechargeable battery. It is appreciated that FIG. 2A represents an exemplary system 10 operating under the “manual system control mode,” whereas FIG. 2B represents another exemplary system 10 operating under the “signal system control mode.” Accordingly, such systems 10 under different control mode include the same members and/or units, although various audiovisual or control signals may then be sent to and received by different members and/or units in each of such control modes.

As briefly described in FIGS. 1A to 1D, the body member 20 includes at least one first unit and at least one second unit, where the first unit defines various parts capable of contacting one or more portions of the pelvic structure when engaging therewith and providing various stimuli through one or more movements thereof. The body parts with various configurations are generally similar to those of the prior art devices, whereas various novel body parts are provided in the co-pending Applications.

The actuator member 60 includes at least one driver unit 61 and at least one stimulator unit 65. The main function of the actuator member 60 is to effect one or multiple movements of one or multiple parts of the body member 20 (i.e., the body parts of the first unit thereof). To this end, the driver unit 61 receives electric power from the power member and generates driving force which is transferred to the stimulator unit 65 through at least one power transmission unit (not included in the figure). The driving unit 61 may be a conventional electric motor for generating rotational movement of its axle, an electromagnetic vibrator assembly for generating reciprocating movement of a mobile assembly. The rotational movement of the axle of the electric motor may be converted by various power conversion units to effect various driving forces. When the system is to effect multiple movements, the actuator member 60 may include multiple driver units 61 each effecting one or more of such movements either independently or in cooperation. The stimulator unit 65 is then arranged to receive the driving force, to optionally convert the driving force into actuating force, and to effect desirable movement of a preset part of the first unit of the body member 20 by such actuating force. To this end, the stimulator unit 65 mechanically, electrically, and/or magnetically couples with the preset part of the body member 20 and ensures its actuating force to effect desirable movement of the body part. The actuator member 60 may also include the power transmission unit which may transmit the driving and/or actuating forces from one to another locations of the body member 20 while maintaining or changing amplitudes and/or directions of the forces. The power transmission unit may be any conventional articles of commerce capable of transmitting the rotational and/or translational movements therealong, and examples of the power transmission unit may include, but not be limited to, assemblies of multiple axles coupled by one or more universal joints, rigid or flexible metal cables, and the like. Such an actuator member 60 may also include one or multiple driver and/or stimulator units 61, 65 which may be operatively coupled to each other in various modes. In one example, a single driver unit 61 may be operatively coupled to a single stimulator unit 65 and deliver such driving force thereto. In another example, a single driver unit 61 may be operatively coupled to two or more stimulator units 65 and deliver the same driving force to multiple stimulator units 65 one at a time or simultaneously. The actuator member 60 may also include one or more power conversion units for delivering different driving forces to different stimulator units 65. In another example, two or more driver units 61 may be operatively coupled to a single stimulator unit 65 and deliver different driving forces thereto one at a time or simultaneously. When desirable, at least two of the driver units 61 may be arranged to drive the stimulator unit 65 simultaneously so as to effect compound actuating force.

Similarly, the actuator member 60 may be coupled to the body member 20 in various modes. In one example, a single stimulator unit 65 may operatively couple with a single body part and deliver the driving and/or actuating forces thereto. In another example, a single stimulator unit 65 may operatively couple with two or more body parts and deliver the same driving or actuating forces to multiple body parts one at a time or simultaneously. In another example, multiple stimulator units 65 may operatively couple with a single body part and deliver different driving or actuating forces to such a body part one at a time or simultaneously. When desirable, at least two of the stimulator units 65 may be arranged to move the body part simultaneously so as to effect compound movement of such a part. The actuator member 60 may manipulate various movements of the single part or multiple parts of the body member 20 by its single or multiple stimulator units 65 by various actuating forces. For example, the actuator member 60 may be arranged to generate various types of movements of the single or multiple units (or their parts) of the body member 20. In one example, the actuator member 60 may generate vibration of at least a substantial part of the first unit of the body member 20, vibration of at least one but not all of the head, trunk, and base of the first unit, vibration of one or multiple bulges designated to stimulate the clitoris, G-spot, pelvic wall, and so on. In another example, the actuator member 60 may generate translation of at least a substantial part of such a first unit, translation of at least one but not all of the head, trunk, and base of the first unit, translation of such bulges, and the like, where such translation may be effected through and/or around the pelvic opening for stimulating such clitoris, G-spot, pelvic wall, and the like, and where such translation may be parallel to, perpendicular to or at an angle with respect to the pelvic opening and/or longitudinal axis of the first unit. In another example, the actuator member 60 may generate rotation or pivoting of at least a substantial part of the first unit, rotation or pivoting of at least one but not all of the head, trunk, and base of such a first unit, rotation or pivoting of such bulges, and the like. It is appreciated that such rotation or pivoting may be effected about the longitudinal axis of the first unit or, alternatively, may be effected about a center of rotation or an axis of rotation which may be formed in the first or second units of the body member 20. Therefore, such rotation or pivoting may be effected to the clitoris, G-spot, and pelvic wall when desirable. In addition and depending on the location of such a center, the rotation or pivoting may be effected as swivelling of an entire or only a part of the first unit. In another example, the actuator member 60 may generate tapping or thumping of at least a substantial part of the first unit, tapping or thumping of at least one but not all of the head, trunk, and base of the first unit, tapping or thumping of the bulges, and the like. Therefore, the actuator unit 60 may provide such tapping or thumping stimuli to the clitoris, G-spot, and pelvic wall. In another example, such an actuator member 60 may generate deformation of at least a substantial part of the first unit, deformation of at least one but not all of such head, trunk, and base of the first unit, deformation of one or more of the bulges, and so on. Other configurational details of the body and actuator members 20, 60 for effecting the above movements are provided in the first of the co-pending Applications.

The control member 70 may include at least one control unit 77 and at least one of at least one input unit 71 and sensor unit 75. The main function of the control member 70 is to generate signals for manipulating various units of the actuator member 60. To this end, the input and/or sensor units 71, 75 may be arranged to receive various user inputs and to generate sensing signals in response thereto, while the control unit 77 may be arranged to generate control signals according to the sensing signals such that the actuator member 60 may generate the driving and/or actuating forces in response to the user inputs. Accordingly, the stimulator units 65 of the actuator member 60 may actuate the body part in order to deliver desired stimuli to a single or multiple portions of the pelvic structure. As have been disclosed in the first of the co-pending Applications, the input and sensor units 71, 75 basically refer to similar articles capable of receiving various user inputs and generating various sensing signals in response thereto. Within the scope of this invention, those articles incorporated into the first unit of the body member are to be referred to as the sensor units 75, whereas those incorporated into the second unit of the body member will be referred to as the input units 71, unless otherwise specified. Other configurational details of the input and sensor units 71, 75 of the control member 60 as well as further control operations of the control member 60 have been disclosed in the first of the co-pending Applications and will be omitted for ease of illustration.

As mentioned above, the control member 70 may include at least one internal audiovisual input unit (or to be abbreviated as an “A/V input unit” hereinafter) 81, at least one internal audiovisual output unit (or to be abbreviated as an “A/V output unit” hereinafter) 83, at least one storage unit 85, at least one signal processing unit 87, and other units such as, e.g., a driver unit. The A/V input unit 81 may have any shapes and/or sizes and any number of such units 81 may be incorporated into the system 10 as long as the A/V input unit 81 may obtain the audiovisual (or control) signals (to be abbreviated as “A/V or control signals” hereinafter) through various sources of signals, images, and/or sounds as described above. When desirable, the A/V input unit 81 may operatively couple with microphones or cameras of external devices and import the A/V or control signals directly therefrom through wire or wirelessly. The control member 70 may include different A/V input units 81 to obtain different types of A/V or control signals, sounds, and/or images. Such an A/V input unit 81 is generally disposed in the second unit of the body member 20, although such may be disposed in the first unit as well. The A/V output unit 83 may have any shapes and/or sizes and any number of such units 83 may be included in the system 10 as far as the A/V output unit 83 may generate sounds and/or images based on the A/V or control signals obtained from such sources of signals, images, and/or sounds as described above. When desirable, the A/V output unit 83 may also be operatively coupled to speakers and/or displays of external devices and export such A/V or control signals, sounds, and/or images directly thereto by wire or wirelessly, thereby generating such sounds with the external speakers and/or such images by the external displays. In addition, the control member 70 may have different A/V output units 83 to generate different types of sounds and/or images.

The storage unit 85 may store various A/V or control signals temporarily or permanently. Such an unit 85 may be provided from any conventional data storage articles such as, e.g., magnetic tapes, magnetic disks, optical disks, semiconductor chips, and other data storage devices for storing analog and/or digital data therein. Depending upon types of the articles, the control member may also include suitable drivers to operate the storage unit 85, where examples of the drivers may include, but not be limited to, magnetic or optical tape drivers, magnetic or optical disk drivers, circuits for recognizing and retrieving desired signals, and the like. Such signals may be classified in various modes, e.g., based upon contents of such signals, their classifications, presence or absence of the action bases therein, source thereof, their voice bases, and the like. Therefore, the control member 70 may readily find and then retrieve the desired signal from such a storage unit 85. The storage unit 85 may be disposed in various locations of the system 10 and may be exposed or hidden in the first and/or second units of the body member 20. When desirable, the storage unit 85 may be provided as a replaceable cartridge such that the user may load a desired storage unit 85, change such a unit 85 full of capacity, and the like. The control member 70 may also be arranged to communicate with external storage article and to send, store, search, and/or retrieve desired signals thereto or therefrom.

It is to be understood within the scope of the present invention that any A/V signal is deemed to define at least one of a content basis, a voice basis, an action basis, and a background basis. The content basis refers to any word or phrase of the user or third party which may or may not carry any meaning, and the voice basis means wave characteristics of the audio signal of the user or third party characterizing audible waves generated by human vocal cords and/or those produced by mechanical equivalents of the vocal cords. In contrary, the action basis represents characteristics of the sound accompanying actions of the user or third part and may contain neither the content basis nor the voice basis. Finally, the background basis means none of the above such as, e.g., music, melody, and the like. It is appreciated that non-instrumental music such as opera may be deemed as sound composed of audio signals with the voice basis superposed to the background basis. With these definitions, the signal processing unit 87 receives the A/V signals and modifies such A/V signals by changing, e.g., at least on temporal pattern thereof, their amplitudes, their frequencies, their orders, and so on. Such a processing unit 87 may also generate compound signals by combining multiple A/V signals or, in the alternative, may generate synthesized signals by changing at least one basis of the A/V signals, and the like.

As shown in FIGS. 2A and 2B, the control unit 77 may receive the first A/V or control signals through the A/V input unit 81 and sends the second A/V or control signals to the A/V output unit 83. It is appreciated that the control unit 77 may send to the A/V output unit 83 the same first A/V or control signals received thereby from the A/V input unit 81 so that the first and second A/V or control signals may become identical. In the alternative, the control unit 77 may receive the first A/V or control signals and send different A/V or control signals to the A/V output unit 83 so that the second signals may be different from the first signals. In the latter embodiment, the control unit 77 may also change at least a portion of such first signals with or without using the signal processing unit 87, may retrieve different signals from the storage unit 85 based upon the first signals and then send the retrieved signals to the audio output unit 83, and the like.

In contrary to such A/V input and output units 81, 83, the storage and processing units 85, 87 may send various A/V or control signals to the control unit 77, and receive the same or different A/V or control signals from the control unit 77. That is, the storage and processing units 85, 87 may effect the control unit 77 and control member 70 so that the control unit 77 or member 70 may manipulate the actuator member 60 and/or body member 20 in different mechanisms to effect different movements of a single or multiple parts of the body member 20, to generate different stimuli by the same or different parts of the body member 20, to provide various stimuli onto a single portion or multiple portions of the pelvic structure, to generate different sounds and/or images in response to various user inputs and/or movements of the body parts, and the like. Various control mechanisms of such a control unit 77 and member 70 will now be described in greater detail below.

First, the control member 70 may obtain the A/V or control signals through various sources. In one example and as described above, the control member 70 may retrieve pre-recorded signals from the storage unit 85. When the retrieved or internal signals are A/V signals, the A/V output unit 83 may directly play such signals so as to generate the sounds and/or images. When the internal signals are control signals, the A/V output unit 83 may generate the sounds and/or images while controlling one or more bases of such sounds and/or images based on such internal control signals. It is appreciated that the storage unit 85 may store therein such A/V or control signals which may be analog or digital signals and that such an unit 85 may store the signals with or without data compression. The stored signals may be related to the user or third party. In another example, the control member 70 may be operatively coupled to external data storage devices so as to retrieve the pre-recorded A/V or control signals therefrom. When desirable, the control member 70 may include appropriate drivers to operate the data storage devices or, alternatively, may be arranged to manipulate external drivers to operate the data storage devices. In another example, the control member 70 may obtain such A/V or control signals from various external A/V devices. Such signals may generally be pre-recorded signals and may also be related to the user and/or third party. In another example, the control member 70 obtains such A/V or control signals from various external communication devices. Such signals may be pre-recorded signals or live signals and related to the user or third party. In another example, the control member 70 may receive the pre-recorded or real-time A/V or control signals of the user or third party through an internet. The control member 70 may download such signals and then manipulate the A/V output unit 83 based thereon or may directly generate the sounds and/or images in response thereto in real time. In another example, the control member 70 may generate such compound or synthesized signals from multiple A/V or control signals in order to generate the sounds and/or images therefrom. Once obtaining the A/V or control signals, the control member 70 may then process the A/V or control signals and generate the desired sounds and/or images based upon the procedures exemplified in the first example of this paragraph. It is to be understood that such A/V or control signals may be analog or digital signals and may also be compressed when desirable.

Secondly, the control member 70 may turn on or activate the A/V output unit 83 and generate the sounds and/or images based upon activation of the system 10 and/or its various members. In one example, the control member 70 may turn on the A/V output unit 83 while the system 10 is in use, as may be signified when a main switch is turned on, when the actuator member 60 or its stimulator units 65 are activated, and so on. The control member 70 may further activate the A/V output unit 83 when one or more specific input and/or sensor units 71, 75 receive a specific user input or any user inputs through a preset or any area thereof, when one or more dynamic patterns of such user input reaches or exceeds a preset threshold, and the like. The control member 70 may also activate the A/V output unit 83 when the first unit of the body member 20 is engaged with the pelvic structure, when at least a preset length of the first part is inserted into the internal cavity, when the user generates a motion or movement of the entire system 10 beyond a preset acceleration, velocity, displacement, and the like.

The control member 70 may also manipulate the A/V output unit 83 to generate specific sounds and/or images for a preset period of time using a single or multiple A/V signals in a random order or in a preset order, to generate different sounds and/or images for a preset period of time in a random or preset order, and in other manners as will be described in greater detail below, in response to various user inputs, A/V or control signals, and/or other signals. In one example, the control member 70 may generate such sounds and/or images in a manner based upon the user input (or its dynamic pattern) applied to the input unit 71 or sensor unit 75. Therefore, the user or third party may curtail their input in such a way to effect desired sounds and/or images in the desired mode. In another example, such a control member 70 may generate the sounds and/or images in such a manner based on one or more dynamic features of movement of a single part or multiple parts of the body member 20. Accordingly, the A/V output unit 83 may generate different sounds and/or images depending upon, e.g., a speed of vibration of the body parts, a displacement distance of the first unit, and the like. In another example, the control member 70 may generate such sounds and/or images in a manner based upon the state of the system 10, whether the system 10 is turned on or off, whether the actuator or control member 60, 70 is turned on or off, and the like. In another example and when the system 10 have multiple actuator members 60 and/or stimulator units 65 and/or when the body member 20 defines multiple parts, such a control member 70 may also generate such sounds and/or images in a way based on which actuator member 60, stimulator unit 65, and/or part may be selected by the user inputs for effecting a single or multiple movements. When the actuator members 60 or their stimulator units 65 may generate multiple different movements, the control member 70 may further generate such sounds and/or images in such a manner depending on which movement may be selected by the user input. In another example, the control member 70 may generate such sounds and/or images in such a manner based upon states of engagement of the system 10 with the pelvic structure. Accordingly, the system 10 may activate the A/V output unit 83 and generate the sounds and/or images as a preset part of the first unit is inserted into the internal cavity, a preset part of the first unit engages with the clitoris, and the like. In another example, the control member 70 may generate such sounds and/or images in a manner based upon a type or extent of movement of the system as a whole so that the A/V output unit 83 may be activated so as to generate the same sounds and/or images in different patterns, to generate different sounds and/or images in response to various dynamic features of the movement of the single or multiple parts of the body member 20, and the like. In another example, the control member 70 may activate the A/V output unit 83 and manipulate such an unit 83 to generate the sounds and/or images in such a manner in response to the user input carried in the voice of the user or third party. As long as such a control member 70 may recognize such an audible user input, such a member 70 may also perform any of the above and following control mechanisms from the audible user inputs. In another example, the control member 70 may also manipulate the A/V output unit 83 based upon the external A/V or control signals which may be pre-recorded or real-time and which may delivered to the control member 70 through the external A/V or communication devices, internet, and the like.

Such a control member 70 may also generate the compound and/or synthesized A/V or control signals and generate the sounds and/or images therefrom in such a manner. Accordingly, the control member 70 may play the sounds and/or display the images defining only one of such content, voice, action, and background bases, another sound and/or image having two or more of the content, voice, action, and background bases, and the like. The control member 70 may also be arranged to change only one basis of such signals and to play such modified signals for generating such sound or image, to impose at least one basis of first signals to second signals as described above, and so on. Such a control member 70 may also generate the synthesized sound by altering only one (or multiple) basis of the signals but not all bases thereof. The control member 70 may perform such synthesis through the signal processing unit 87.

The control member 70 preferably synchronizes various sounds and/or images generated by the A/V output unit 83 with numerous events such as, e.g., various operational states of the system 10, its members, and/or its units, various dynamic features of the movements of the single or multiple parts of the body member 20, various dynamic patterns of the user input, and so on. For example, the sound and/or image generated by the A/V output unit 83 may be temporarily synchronized with such events so that such an unit 83 generates the sound and/or image simultaneously with at least one of such events, immediately after at least one of such events, in a preset interval after at least one of the events, and the like. Similarly, such an unit 83 may be arranged to generate such sound and/or image as far as at least one of such events persists, to stop generating such sound or image simultaneously with cessation of at least one of such events, immediate after cessation of at least one of the events, in a preset interval after cessation of at least one of such events, and the like. Alternatively, the A/V output unit 83 may stop generating such sound and/or image only after it receives another user input. In addition, the event actuating the A/V output unit 83 may not be the same event terminating such an unit 83. Therefore, the A/V output unit 83 may start to generate the sound with one event and stop to generate such sound and/or image with another event. In another example, the sounds and/or images generated by the A/V output unit 83 may be spatially synchronized with such events so that the A/V output unit 83 may generate the sounds and/or images as the user input is applied to the input and/or sensor units in a specific spatial pattern, when such events may occur in a specific spatial mode, and the like.

Moreover, the control member 70 may generate such sounds and/or images based on various A/V or control signals obtained through various sources. In one example, the A/V output unit 83 may generate the sounds and/or images based on internal A/V or control signals stored in the storage unit 85 and retrieved by the control member 70. In another example, the A/V output unit 83 may generate the sounds and/or images based upon the A/V or control signals supplied thereto by the user in real time, either in the form of the user input or as a verbal (or voice) command. In another example, such an unit 83 may generate the sounds and/or bases based upon various A/V or control signals obtained from the external communication or audio devices or from the internet, whether such signals may be pre-recorded ones or real-time ones. In another example, the A/V input unit 83 may also generate the sounds and/or images based on the compound and/or synthesized signals obtained from processing multiple A/V or control signals.

The control member 70 may also manipulate the A/V output unit 83 to provide various stimuli to various portions of such a pelvic structure. In one example, the A/V output unit 83 may be arranged to emit the acoustic waves of the sound directly onto the portion of the pelvic structure so that such a portion may receive such acoustic stimuli therefrom. In another example, the A/V output unit 83 may include at least one vibrating or otherwise moving plate, emit the acoustic waves of such sound onto the plate, and generate vibration or movement of the plate. By disposing the A/V output unit 83 and its plate close to the portion of the pelvic structure, another stimuli may be provided to the portion of such a pelvic structure. It is to be understood that characteristics of such stimuli may depend upon detailed mechanical configuration of such a plate. In another example, the control unit 70 may include at least one auxiliary driver unit capable of generating an auxiliary movement of at least one part of the body member 20. Such auxiliary movement may be delivered to the desired portion of the pelvic structure independently or may be superposed onto the movement of the part effected by the actuator member 60 or its stimulator unit 65.

Furthermore, the control member 70 may generate various sounds and/or images for a preset period of time by manipulating its various units. In one example, the control member 70 may control the A/V output unit 83 to repeat a preset A/V signal or a set of A/V signals during the period. In another example, the control member 70 may control the A/V output unit 83 to play and repeat, as necessary, one or more A/V signals randomly, in a preset order, or in a variable order as determined by the user input or other events as described above. In another example, the control member 70 may control the A/V output unit 83 to play and to repeat, as necessary, different A/V signals based upon the external A/V or control signals which may be pre-recorded or real-time, which may be related to the user or a third party, and which may be provided from the external A/V and/or communication devices or from the internet. In another example, such a control member 70 may control the A/V output unit 83 to play different A/V signals during the period depending on the user input supplied to the input unit 71 and/or sensor unit 75, based on the dynamic patterns of the user input, depending on the dynamic features of the movements of the body part, based on the movement of the entire system effected by the user or a third party manually, based upon various operational states of the system 10 or its members as described above, and the like. The control member 70 may categorize multiple A/V or control signals based on their content, voice, action, and/or background bases and play such signals from a specific category or group during the period, play such signals from different groups depending upon various events as mentioned above, and the like.

In addition, the control member 70 may also receive such A/V or control signals from various sources as described herein and then control various operations of the system 10 according thereto. For example, the control member 70 may control the dynamic features of the movements of the single part or multiple parts, number of the body parts recruited for the movements, and the like. The control member 70 may also control the temporal and/or spatial patterns of the sounds and/or images based on such A/V or control signals.

The pelvic relaxing system of the present invention may further be construct to be waterproof. For example, various input units of the body member may be covered by a waterproof layer or may be disposed inside the second unit of the body member. In addition, the system may be arranged to run by a rechargeable battery which may be recharged by electromagnetic induction from outside.

Unless otherwise specified, various features of one embodiment of one aspect of the present invention may apply interchangeably to other embodiments of the same aspect of this invention and/or embodiments of one or more of other aspects of this invention. Therefore, various external A/V input and/or output units of the above embodiment may be interchangeably used in conjunction with any of the above pelvic relaxing systems as well as those of the co-pending Applications. In addition, such a system may include one or multiple input and/or sensor units of the same or different types, may also incorporate therein one or multiple internal A/V input and/or output units of the same or different types, and the like.

Unless otherwise specified, various features of one embodiment of one aspect of the present invention may further apply interchangeably to other embodiments of other aspects of other inventions disclosed in the co-pending Applications. Accordingly, such input and/or sensor units of this invention may be used in any pelvic relaxing systems including various clitoris and/or G-spot stimulators, those including dynamic control capabilities, those incorporated with interactive capabilities, those including electric stimulators, those incorporating adjustable body members, those with retention mechanisms, those with feedback mechanisms, those with reciprocating body members, and the like.

The pelvic relaxing systems of this invention offer various advantages over their conventional counterparts. First of all, such a system allows the user to manually or automatically generate sounds and/or images while using the system. Therefore, the user may play the desired sounds and images while using the system and providing various stimuli to her pelvic structure, thereby obviating the user from imaging voices from and/or images of a desired partner. Secondly, such a system may play the sounds and images in synchronization with various operational characteristics thereof. Accordingly, such sounds and/or images may increase or decrease in its amplitudes, change its speed and/or tone, vary its content bases, voice bases, and/or action bases in response to the user input, movements of the entire system, physical or mental states of the user, and the like. In addition, the system may also obtain or generate such sounds and/or images and manipulate various operational characteristics of the system according thereto. For example, various dynamic features of various movements of one or multiple body parts may be manipulated based upon, in response to or in synchronization with the sounds and/or images, the body parts may also engage with different portions of the pelvic structure based upon such sounds and/or images, and so on. Moreover, the system may generate and play the synthesized audiovisual signals using different audiovisual (or control) signals, thereby allowing the user to play such sounds carrying the voice of the desired partner and/or such images of the partner, carrying the desired content, and so on. The system may generate the sounds and/or images while fulfilling various advantages of other pelvic relaxing systems disclosed in the co-pending Applications.

The above systems, methods, and/or processes of the present invention may be applied to or utilized for various purposes. As described above, such systems, methods, and/or processes may be used to play the sound while providing various stimuli to various portions of such a pelvic structure including the clitoris, G-spot, and other portions on the pelvic wall. In addition, the systems, methods, and processes of the present invention may be applied to other pelvic relaxing systems such as, e.g., systems with various clitoris and/or G-spot stimulators, systems with interactive capabilities, systems synchronized with internal and external signals, systems including electric stimulators, systems with adjustable body members, systems with retention mechanisms, systems with feedback mechanisms, systems having reciprocating body members, systems controlled by various input and/or sensor units capable of receiving the user input without requiring the user to change the grip, and the like.

It is appreciated that the Disclosure Documents which have been referred to in the section of “Cross-Reference” and bear the Serial Numbers 611,027, 611,023 and 611,331 have been referred to herein as the “co-pending applications.”

It is to be understood that, while various aspects and embodiments of the present invention have been described in conjunction with the detailed description thereof, the foregoing description is intended to illustrate and not to limit the scope of the invention, which is defined by the scope of the appended claims. Other embodiments, aspects, advantages, and modifications are within the scope of the following claims.

What is claimed is:

1. A pelvic relaxing system for providing tactile stimuli onto at least a portion of a pelvic structure through a plurality of movements of at least one part of said system based on at least one audiovisual signal, wherein said pelvic structure includes an entry and a wall, wherein said entry is configured to form an orifice therethrough, and wherein said wall is configured to include muscles and to define an internal cavity which extends inwardly from said entry and which is also bound by said muscles, said system comprising:
at least one body member which is configured to include a first unit with at least one first part for contacting said portion of said pelvic structure when engaged therewith and to include a second unit for providing a grip to an user;
at least one actuator member which is configured to effect said movements of said first part each defining at least one dynamic feature and to provide each of said stimuli to said portion of said structure through each of said movements; and
at least one control member which is configured to obtain said signal from at least one source, and to control said actuator member for effecting said movements of said first part while manipulating said dynamic feature of each of said movements based on said obtained signal,
whereby said system is configured to effect each of said movements of said first part without requiring said user to manually move said body member to effect said different movements.

2. The system of claim 1, wherein said control member is configured to include at least one of an input unit and a sensor unit both of which are configured to receive an user input from said user.

3. The system of claim 2, wherein said input unit is configured to be disposed in said first unit and to receive said user input which is applied thereto by a hand of said user, whereas said sensor unit is configured to be disposed in said second unit and to receive said user input which is applied thereto through said pelvic structure.

4. The system of claim 3, wherein said user input has at least one dynamic pattern and wherein said control member is configured to control at least one of said actuator and control members based upon said dynamic pattern of said user input.

5. The system of claim 1, wherein said control member is configured to obtain a plurality of said signals and to manipulate said actuator member for effecting said movements of said first part at least two of which define different dynamic features based on said signal one of simultaneously and one at a time.

6. The system of claim 1 further comprising a main switch capable of moving between a plurality of states each of which is designated for a different operation of said actuator member, wherein said control member is configured to obtain said signal based on said state and to manipulate said dynamic feature of said movement of said first part based upon said obtained signal.

7. The system of claim 1, wherein said control member is configured to obtain said signal from at least one of an internet, external audiovisual device, and external communication device.

8. The system of claim 1, wherein said control member is configured to obtain said signal from at least one of a verbal command and a bodily command of said user.

9. The system of claim 1, wherein said control member is configured to monitor engagement of said first part of said body member with said pelvic cavity and to manipulate said dynamic feature of said movement of said first part based upon said obtained signal.

10. The system of claim 9, wherein said control member is configured to obtain one of a plurality of said signals based upon at least one of an extent and a type of said engagement and to manipulate at least one of said actuator and body members based upon said signal.

11. The system of claim 1, wherein said control member is also configured to monitor at least one physiological variable of said pelvic structure, to obtain one of a plurality of said signals based upon at least one of an extent and a type of said engagement, and to manipulate at least one of said actuator and body members based on said signal.

12. The system of claim 1, wherein said control member is configured to obtain a first audiovisual signal defining a first content basis and a first voice basis from a plurality of said signals, to obtain a second audiovisual signal having a second content basis and a second voice basis from said plurality of said signals, to generate at least one synthesized signal by replacing one of said bases of one of said audiovisual signals by said one of said bases of another of said audiovisual signals, and then to manipulate at least one of said body and actuator members based on said synthesized signal.

13. The system of claim 1, wherein said control member is configured to obtain a first audiovisual signal defining a preset content basis and a preset voice basis from a plurality of said signals which are one of pre-recorded and real time and related to one of said user and a third party, and to alter at least one of said bases of said first signal, thereby generating a synthesized signal, and to manipulate at least one of said body and actuator members based on said altered signal.

14. The system of claim 1, wherein said system includes a plurality of actuator members each of which is configured to effect each of said movements of said first part defining said dynamic feature for providing said stimuli to said portion of said pelvic structure through said movement, and wherein said control member is configured to obtain said signal, to select one of said actuator members based on said signal, and to manipulate said dynamic feature of said movement of said first part effected by said selected actuator member based upon said signal.

15. The system of claim 1, wherein said body member is configured to include a plurality of first parts each for contacting said portion of said pelvic structure when engaged therewith, wherein said actuator member is configured to effect at least one movement of each of said first parts with at least one dynamic feature for providing said stimuli to said portion of said structure through said movement, and wherein said control member is configured to obtain said signal from at least one source, to select at least one of said parts based upon said signal, and then to manipulate said dynamic feature of said movement of said selected part based upon said signal.

16. The system of claim 1, wherein said obtained signal is configured to also define at least one of a content basis, a voice basis, and an action basis, and wherein said control member is configured to manipulate said dynamic feature of said movement of said first part based upon at least one of said bases of said obtained signal.

17. The system of claim 1, wherein said control member is configured to effect said movement of said first part as a response to dynamic pattern of an user input, to obtain from at least one source at least one control signal which is configured to include neither a content basis nor a voice basis, and to manipulate said dynamic feature of said movement of said first part based on said obtained control signal.

18. A pelvic relaxing system for providing tactile stimuli onto at least a portion of a pelvic structure based upon at least one of a sound and an image, wherein said pelvic structure includes an entry as well as a wall, wherein said entry is configured to form an orifice therethrough, and wherein said wall is configured to have muscles and to define an internal cavity which is configured to extend inwardly from said entry and to be further bound by said muscles, said system comprising:
at least one body member which is configured to include a first unit with at least one first part for contacting said portion of said pelvic structure when engaged therewith and to include a second unit for providing a grip to an user;
at least one actuator member which is configured to effect a plurality of movements of said first part each defining at least one dynamic feature and to provide each of said stimuli to said portion of said structure through each of said movements; and
at least one control member which is configured to obtain said signal from at least one source, and to control said actuator member for effecting said movements of said first part while manipulating said dynamic feature of each of said movements based on said obtained signal and while outputting at least one of said sound and image.

19. The system of claim 18, wherein said control member is configured to obtain a first audiovisual signal defining a preset content basis and a preset voice basis from a plurality of said signals which are one of pre-recorded and real time and related to one of said user and a third party, and to alter at least one of said bases of said first signal, thereby generating a synthesized signal, and to manipulate at least one of said body and actuator members based on said altered signal.

20. A method of obtaining at least one audiovisual (or control) signal and for providing tactile stimuli effected by at least one part of a body member of a pelvic relaxing system onto at least a portion of a pelvic structure at least partially based on said signal, wherein said pelvic structure includes an entry and a wall, wherein said entry forms therethrough an orifice, and wherein said wall includes muscles and defines an internal cavity which extends inwardly from said entry and is bound by said muscles, said method comprising the steps of:
storing a plurality of said signals which are pre-recorded;
obtaining at least one signal from said stored signals;
engaging said body member with said pelvic structure; and
manipulating at least one movement of said part of said body member based on (or in at least partial synchronization with) said signal.
